(19) United States
(12) Patent Application Publication
(10) Pub. No.: US 2026/0117191 A1
(43) Pub. Date: Apr. 30, 2026

(54) MAGNETICALLY DIRECTED ASSEMBLY OF PATTERNED CAPILLARY BEDS

(71) Applicant: The Regents of the University of Michigan, Ann Arbor, MI (US)

(72) Inventors: Brendon M. BAKER, Ann Arbor, MI (US); Abdon PENA-FRANCESCH, Ann Arbor, MI (US); Harrison L. HIRAKI, Ann Arbor, MI (US); Maggie E. JEWETT, Columbus, OH (US)

(73) Assignee: The Regents of the University of Michigan, Ann Arbor, MI (US)

(21) Appl. No.: 19/482,505

(22) PCT Filed: May 9, 2024
(86) PCT No.: PCT/US2024/028607

§ 371 Date: Nov. 7, 2025

Related U.S. Application Data
(60) Provisional application No. 63/465,049, filed on May 9, 2023.

(51) Int. Cl. C12N 5/071

(52) U.S. Cl. CPC C12N 5/069; C12N 2531/00; C12N 2533/30; C12N 2533/52; C12N 2533/54

(57) ABSTRACT

The present disclosure provides a method of producing a cellular support material. The method may include fabricating a microfiber lattice having a three-dimensional structure including a plurality of fibers and a plurality of voids distributed between the plurality of fibers. At least a portion of the plurality of fibers includes at least one first magnetic particle at least partially embedded therein. The method includes introducing cells to the microfiber lattice. At least a portion of the cells includes at least one second magnetic particle at least partially embedded therein. The method includes associating the cells with the plurality of fibers within the microfiber lattice. The method further includes encapsulating the microfiber lattice and associated cells with a hydrogel. The method further includes removing at least a portion of the plurality of fibers.

### CROSS-REFERENCE TO RELATED APPLICATIONS

This application claims the benefit of U.S. Provisional Application No. 63/465,049, filed on May 9, 2023. The entire disclosure of the above-mentioned application is incorporated herein by reference.

### GOVERNMENT SUPPORT

This invention was made with government support under EEC1647837 awarded by the National Science Foundation. The government has certain rights in the invention.

### FIELD

The present disclosure relates to the formation of capillary beds including fabricating temporary microfiber scaffold lattices and a magnetization technique to efficiently and uniformly seed lattices with cells.

### BACKGROUND

This section provides background information related to the present disclosure which is not necessarily prior art.

The presence and organization of the microvasculature ensures the proper function of most tissues and organs. Capillaries, the smallest length-scale of the microvasculature, most directly enable transport to and from tissue parenchyma due to their high surface area to volume ratio and sheer abundance. The organization and density of capillary beds vary widely across tissue systems, ranging from the random, sparse networks within adipose tissue to the dense and highly aligned arrays in the myocardium. In particular, the high transport demands of metabolically active tissues such as myocardium necessitates a high density of capillaries where each aligned cardiomyocyte bundle is juxtaposed with one or more co-aligned capillaries. The creation of 3D cardiac tissues at relevant scales for replacement therapies has been in part hampered by the inability to integrate dense, organized capillary beds into these metabolically demanding tissues.

Technologies for creating of 3D cardiac tissues have been used for larger scale vessels (e.g., a vessel having a diameter that is greater than 50 micrometers). While there have been many recent advances in tissue engineering larger-scale vessels across the entire vascular tree, the rapid fabrication and integration of organized microvessel networks at capillary scale with parenchymal tissues has proved challenging.

There have been many recent advances across a diversity of approaches to engineering microvasculature. Bottom-up approaches include angiogenesis and vasculogenic assembly, while top-down approaches include 3D printing with sacrificial inks and two photon mediated photo-ablation of hydrogels to generate patent channels. Bottom-up approaches rely on cell-directed assembly which generally achieves capillary-scale architecture but is often challenging to scale up to therapeutically relevant length-scales. In several approaches to drive angiogenesis into parenchymal cell-laden hydrogels, functional microvessels can be generated but are typically disorganized and also take considerable time to form. Similarly, directing vasculogenic assembly can achieve an appropriate size of perfusable 3D capillary networks but are also unorganized, take significant time to assemble, and require support from other cells such as fibroblasts or mesenchymal stem cells to assemble. Alternatively, top-down approaches facilitate highly controlled vascular architectures and generally result in faster assembly of vessels but are unable to generate capillary-scale structures. Specifically, 3D printing approaches using sacrificial inks to print patent fluidic channels are rapid, highly tunable, and can generate organized microchannel architectures but lack the resolution to generate capillary-scale networks. Other techniques such as photodegradation of hydrogels can achieve generate capillary-scale channels, but photo-degradation may harm encapsulated cells and more importantly, capillary-scale channels (5-20 μm diameter) cannot be subsequently seeded with endothelial cells (ECs) via flow-mediated seeding as the diameter of an EC in suspension (>20 μm) exceeds the diameter of the channel. Notably, Arawaka, et al. were able to pattern channels in a photodegradable polyethylene glycol-based hydrogel spanning 10-200 μm in diameter but demonstrated EC seeding could only be achieved at or above 45 μm in diameter. Overall, the limited resolution of top-down approaches and the long temporal requirements and limited organization of cell-driven assembly processes motivates the need for new technologies that can template organized capillary-scale vasculature in 3D.

Moreover, efficiently associating or seeding cells to highly porous polymer lattices (i.e., a microfiber lattice including a plurality of voids) is challenging due to the tendency for cells in suspension to rapidly settle before attaching to a porous lattice. Recent work demonstrates magnetized cells and biomaterials can generate complex cell patterning into tubular structures around a cylindrical magnet, attach single cells onto magnetic posts, levitate cells at air-liquid interfaces, improve seeding of magnetic porous lattices, and magnetically align fibers within hydrogels to guide cell spreading and migration. However, it is challenging to use these technologies to effectively seed cells onto microfiber lattices that are subsequently degraded.

It would be advantageous to develop a method to rapidly create an organized microvasculature that can be readily integrated with a variety of hydrogels containing parenchymal and/or endothelial cell type(s) of choice. Moreover, it would be advantageous to develop a method enabling efficient seeing of cells onto microfiber lattices that can subsequently be degraded without impacting mammalian cell viability or function.

### SUMMARY

This section provides a general summary of the disclosure, and is not a comprehensive disclosure of its full scope or all of its features.

In various aspects, the present disclosure provides a method of producing a cellular support material. The method includes introducing cells to a microfiber lattice. The microfiber lattice has a three-dimensional structure comprising a plurality of fibers and a plurality of voids distributed between the plurality of fibers. At least a portion of the plurality of fibers includes at least one first magnetic particle at least partially embedded therein. Further, at least a portion of the cells include at least one second magnetic particle at least partially embedded therein. The method includes associating the cells with the plurality of fibers within the microfiber lattice. The method includes encapsulating the microfiber lattice and associated cells with a hydrogel. The method includes removing at least a portion of the plurality of fibers.

In one aspect, the method includes fabricating a microfiber lattice having a three-dimensional structure including a plurality of fibers and a plurality of voids distributed between the plurality of fibers.

In one further aspect, fabricating the microfiber lattice includes solution electrowriting (SEW) or fiber electropulling (FEP) a fiber precursor material.

In one aspect, the fiber precursor material includes polycaprolactone (PCL).

In one aspect, the plurality of fibers has an average diameter that is greater than or equal to about 5 micrometers to less than or equal to about 100 micrometers.

In one aspect, method further includes, prior to the associating, polarizing the microfiber lattice.

In one aspect, the plurality of fibers is substantially aligned along a first direction and the polarizing the microfiber lattice includes applying a magnetic field to the microfiber lattice in a second direction substantially orthogonal to the first direction.

In one aspect, the plurality of fibers is substantially aligned along a first direction and the polarizing the microfiber lattice includes applying a magnetic field to the microfiber lattice in a second direction substantially parallel to the first directions.

In one aspect, the removing includes enzymatically degrading at least a portion of the plurality of fibers.

In one aspect, the cells include endothelial cells (ECs).

In one aspect, the first magnetic particle and the second magnetic particle are independently selected from the group consisting of: a ferromagnetic microparticle (FMP), a superparamagnetic iron oxide nanoparticle (SPION), or combinations thereof.

In one aspect, the plurality of fibers include polycaprolactone (PCL), the first magnetic particle includes a ferromagnetic microparticle (FMP), the cells include endothelial cells (ECs), and the second magnetic particle includes a superparamagnetic iron oxide nanoparticle (SPION).

In various aspects, the present disclosure provides a cellular support material. The cellular support material includes a plurality of microchannels defined in a hydrogel matrix. A first portion of the plurality of microchannels are aligned in a first direction. The cellular support material further includes a plurality of cells embedded within surfaces defined by the plurality of microchannels. At least a portion of the plurality of cells includes at least one magnetic particle at least partially embedded therein.

In one aspect, the plurality of microchannels of the cellular support material has an average diameter that is greater than or equal to about 5 micrometers to less than or equal to about 100 micrometers.

In one aspect, the hydrogel matrix of the cellular support material includes a polysaccharide, a polypeptide, a synthetic polymer, or combinations thereof.

In one aspect, the hydrogel matrix includes a polypeptide selected from the group consisting of: collagen, fibronectin, gelatin, derivatives thereof, and combinations thereof.

In one aspect, the plurality of cells of the cellular support material include endothelial cells (ECs).

In one aspect, the at least one magnetic particle includes a superparamagnetic iron oxide nanoparticle (SPION).

In one aspect, the present disclosure provides a method of forming a microvasculature. The method includes growing cells on the cellular support material until the microvasculature is formed.

In various aspects, the present disclosure provides a template for forming a cellular support material. The template includes a plurality of microfibers defining a three-dimensional lattice and including a plurality of voids between the respective microfibers. At least a portion of the plurality of microfibers includes at least one magnetic particle at least partially embedded therein. The template includes a plurality of cells associated with the plurality of microfibers. At least a portion of the plurality of cells includes at least one magnetic particle at least partially embedded therein.

In one aspect, the microfibers include polycaprolactone (PCL) and at least a portion of the plurality of microfibers include at least one ferromagnetic microparticle (FMP) at least partially embedded therein.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a flow chart showing a method of making a cellular support material for a microvasculature in accordance with various aspects of the current technology.

FIGS. 2A-2C show a method of making a cellular support material including generating engineered capillary beds via magnetic lattice fabrication and endothelial cell (EC) seeding according to various aspects of the current technology. FIG. 2A is a schematic overview of a fabricating scheme for generating capillary-scale endothelialized microchannels within engineering parenchymal tissues composed of one or more cell types and a hydrogel of choice. FIG. 2B shows a schematic (left) and a photograph (right) of a solution electrowriting (SEW) method for fabricating capillary templating microfiber lattices. FIG. 2C shows a schematic (left) and photograph (right) of a fiber electropulling (FEP) method for fabricating capillary templating microfiber lattices.

FIGS. 3A-3E show production and characterization of microfiber capillary-templating lattices according to certain aspects of the present disclosure. FIG. 3A shows transmitted light images and fiber diameter quantification of FEP (left) and SEW (right) fibers as a function of polymer solution concentration. FIG. 3B shows transmitted light images and fiber diameter quantification of FEP (left) and SEW (right) SPION- and FMP-loaded fibers. FIG. 3C is a fiber diameter quantification of FEP (FIG. 3C(i)) and SEW (FIG. 3C(ii)) SPION and FMP-loaded fibers formed from 25 w/v % polycaprolactone (PCL) with histograms displaying the fiber diameter frequency of fibers loaded with 40 mg mL⁻¹ FMPs. FIG. 3D shows scanning electron microscope (SEM) images of FEP single (i), FEP multi-layer (ii), SEW single (iii) and SEW multi-layer (iv) PCL (25 w/v %) lattices without FMPs. FIG. 3E shows SEM images of (i) FEP single layer and (ii) SEW single layer lattices with 40 mg mL⁻¹ FMPs. n≥14 lattices. All data are mean±SD. *P<0.05.

FIGS. 4A-4D show that the spacing and diameter of microfibers can be controlled during FEP or SEW fabrication according to various aspects of the present disclosure. FIG. 4A shows transmitted light images of FEP produced 25 w/v % PCL+40 mg ml⁻¹ FMPs fiber diameters at various rotating mandrel speeds. FIG. 4B is a quantification of FEP produced 25 w/v % PCL+40 mg ml⁻¹ FMPs fiber diameters at various rotating mandrel speeds. FIG. 4C shows transmitted light images of 25 w/v % PCL+40 mg ml⁻¹ FMPs fiber diameters produced by SEW at various preset fiber distances. FIG. 4D is a quantification of 25 w/v % PCL+40 mg ml⁻¹ FMPs fiber diameters produced by SEW at various preset fiber distances. n≥58 microfibers. All data are mean±SD. *P<0.05.

FIGS. 5A-5F show that EC loading with either SPIONs of FMPs enables cell magnetization with limited impact on cell viability and barrier function according to various aspects of the present disclosure. FIG. 5A is a schematic of a 3D printed magnetization assay plate containing four neodymium cube magnets fixed at set locations. FIG. 5B is an image of hematoxylin-stained 6-well plate plated with variably magnetized ECs positioned over the 3D printed magnetization assay plate. FIG. 5C shows fluorescent images of propidium iodide and Hoechst-stained pre-magnetized ECs seeded above and away from neodymium magnets. FIG. 5D is a quantification of EC magnetization efficiency. FIG. 5E is a quantification of cell viability. FIG. 5F are fluorescent images of EC monolayers loaded with magnetic particles. n=4 fields of view. All data are mean±SD. *P<0.05.

FIGS. 6A-6C show that cell magnetization with an intermediate particle loading concentration does not affect long-term cell proliferative ability or capability of forming VE-cadherin enriched adherens junctions according to various aspects of the present disclosure. FIG. 6A shows fluorescent images of magnetized ECs passaged once or thrice, assayed for proliferation by EdU incorporation and VE-cadherin immunolocalization. FIG. 6B shows quantifications of EdU+ cells and VE-cadherin expression for cells including (i) SPIONS and (ii) FMPs. FIG. 6C shows SEM images of (i) SPIONS, (ii) FMPs, (iii) naive ECs, and of ECs magnetized by endocytic loading with either (iv) SPIONs or (v) FMPs. n=6 fields of view. All data are mean±SD. *P<0.05.

FIG. 7A-7H show that lattices fabricated with pre-polarized FMPs result in the highest degree of EC spreading along PCL microfibers according to certain aspects of the present disclosure. FIG. 7A shows transmitted light timelapse images of ECs magnetically attracting to a FMP-doped PCL fiber (random magnetic polarization) during lattice seeding; arrowheads A (light) indicate locations of FMP aggregates within the PCL fiber; arrowheads B (dark) indicate magnetized cells. FIG. 7B is schematic of magnetic polarization orientations that are (i) random, (ii) orthogonal to a direction of the microfibers, and (iii) parallel to a direction of the microfibers. FIG. 7C is a schematic of the resulting remanence as measured in a vibrating-sample magnetometer and reported in milli-electromagnetic units (memu). FIG. 7D shows fluorescent images of EC seeded on PCL lattices 24 hours after seeding for (i) unpolarized particles, (ii) magnetic polarization in a random orientation, (iii) magnetic polarization in a direction orthogonal to a direction of the microfibers, and (iv) magnetic polarization in a direction parallel to a direction of the fibers; note that PCL fibers are autofluorescent. FIG. 7E is a quantification of EC density along PCL fibers. FIG. 7F is the resulting diameter of endothelialized structures of FIG. 7E. FIG. 7G shows fluorescent images of EC attachment 24 hours after seeding as a function of seeding density. FIG. 7H is the corresponding quantification of EC density along PCL fiber length. n≥6 fields of view. All data are mean±SD. *P<0.05.

FIGS. 8A-8D show that the bacterial lipase-mediated degradation of polymer fibers does not negatively impact endothelial cell viability according to certain aspects of the present disclosure. FIG. 8A shows fluorescent time course images of PCL microfibers upon lipase-mediated PCL degradation within collagen gels. FIG. 8B shows fluorescent time course images of EC viability in the presence of lipase as determined by propidium iodide and Hoechst staining. FIG. 8C is a normalized fluorescent intensity of PCL microfibers upon lipase-mediated PCL degradation within collagen gels. FIG. 8D is a schematic quantification of EC viability in the presence of lipase as determined by propidium iodide and Hoechst staining.

FIGS. 9A-9F show that magnetically assisted capillary templating improves the viability of cardiomyocyte-laden tissues in accordance with certain aspects of the present disclosure. FIG. 9A is a fluorescent microsphere (Ø=4 μm) perfusion through acellular 4 mg mL⁻¹ collagen hydrogels. FIG. 9B shows fluorescent images of EC networks seeded sparsely (left) and densely (right), then embedded in collagen gel and imaged 48 hours after lipase-mediated microfiber sacrifice. FIG. 9C shows single slice confocal images of microvessels expressing podocalyxin localized along apical/lumenal surfaces and VE-cadherin localized to cell-cell junctions; * denotes open lumenal space. FIG. 9D shows fluorescent microsphere (Ø=4 μm) perfusion through GFP EC-seeded channels in 10 mg mL⁻¹ fibrin hydrogels. FIG. 9E is a cell viability quantification of Phospho-Histone H2A.X- and DAPI-stained iPSC-CM-laden fibrin gels cultured for 5 days following PCL degradation. FIG. 9F shows fluorescent images of Phospho-Histone H2A.X- and DAPI-stained iPSC-CM-laden fibrin gels cultured for 5 days following PCL degradation. n≥7 fields of view. All data are mean±SD. *P<0.05.

FIGS. 10A-10B show that bacterial lipase-mediated degradation of microfibers does not negatively impact endothelial cell viability according to various aspects of the present disclosure. FIG. 10A shows fluorescent images of CMTPX-doped PCL lattices over 24 hours in the presence of varying lipase concentrations. FIG. 10B shows DAPI (green) and propidium iodide (red) staining to assess HUVEC viability when exposed to varying lipase concentrations for 24 hours (Note: nuclei of dead cells colocalize both signals and appear yellow).

FIG. 11 shows that 4 micrometer fluorescent bead perfusion illustrates endothelialized channel lumenization according to certain aspects of the present disclosure. FIG. 11 shows fluorescent images of 5 million mL⁻¹ GFP labeled HUVECs and normal human dermal microvascular endothelial (MVECs) on the PCL lattices 3 days after lipase degradation.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges. As referred to herein, ranges are, unless specified otherwise, inclusive of endpoints and include disclosure of all distinct values and further divided ranges within the entire range. Thus, for example, a range of “from A to B” or “from about A to about B” is inclusive of A and B.

Example embodiments will now be described more fully with reference to the accompanying drawings.

In various aspects, the present disclosure provides a cellular support material and methods of producing a cellular support material to seed temporary microfiber scaffold lattices efficiently and uniformly with cells. In certain aspects, the cellular support material is configured to generate scalable capillary networks for engineering microvascularized tissues. The method of producing a cellular support material according to various aspects of the present disclosure is capable of rapidly generating scalable capillary networks with user-defined architecture integrated with 3D cell-laden hydrogel constructs.

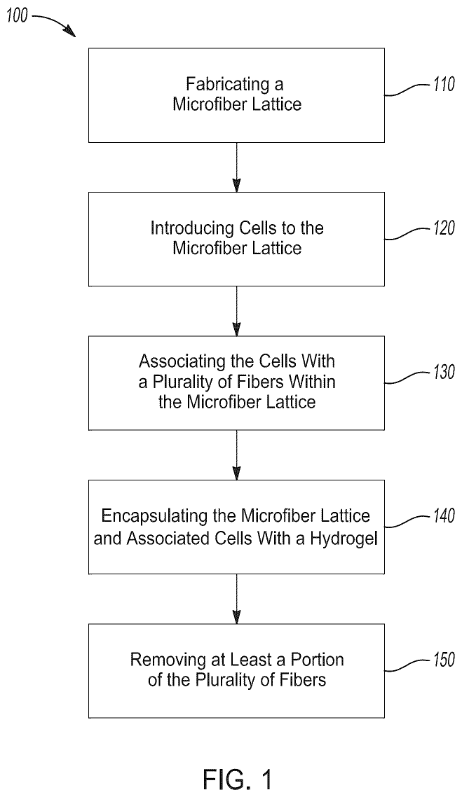

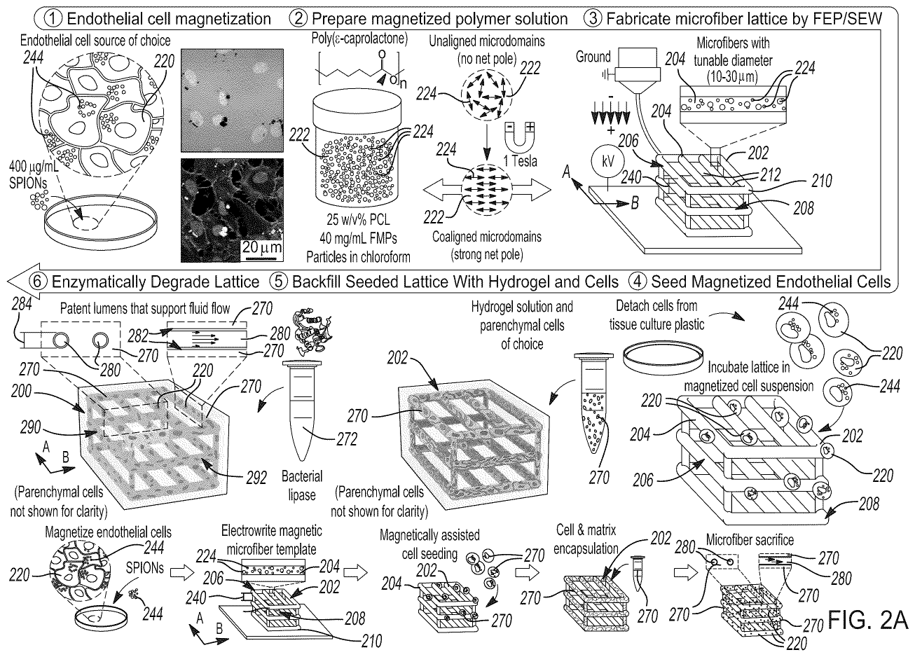

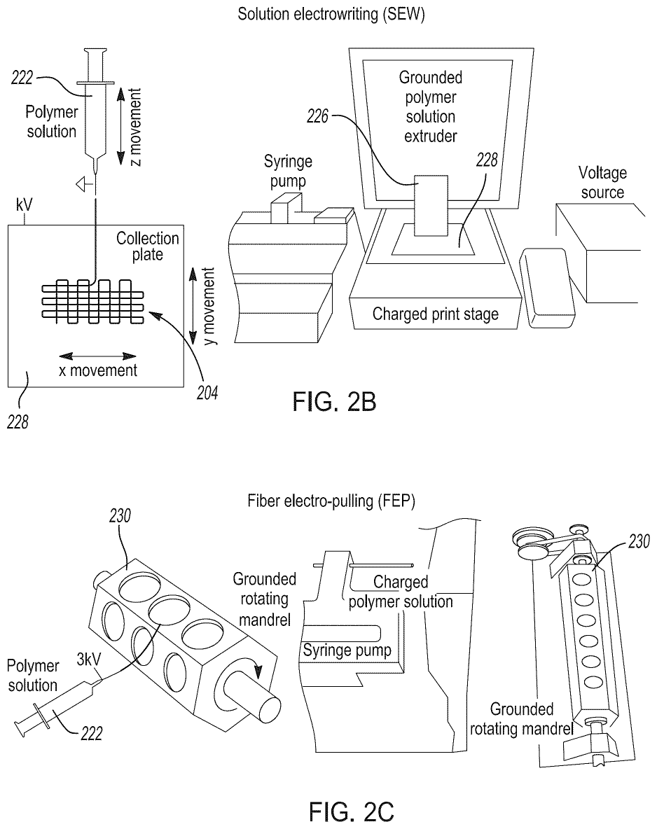

With reference to FIG. 1, a method 100 of producing a cellular support material is provided. In certain aspects, the method 100 includes fabricating a microfiber lattice at 110 (e.g., fabricating a template structure), introducing cells to the microfiber lattice at 120, associating the cells with the plurality of fibers within the microfiber lattice at 130, encapsulating the microfiber lattice and associated cells with a hydrogel at 140, and removing at least a portion of the plurality of fibers at 150. As will be described in greater detail below in the discussion accompanying FIG. 2A-2C, at least a portion of the plurality of fibers of the microfiber lattice include at least one first magnetic particle at least partially embedded therein and at least a portion of the cells include at least one second magnetic particle at least partially embedded therein.

By degrading at least a portion of the microfiber lattice, channels (e.g., microchannels) are formed in the cellular support material. In certain aspects, after the channels are formed, the cellular support material is capable of growing cells to form an engineered tissue. For example, the cellular support material may be utilized to generate capillary-scale endotheliazed microchannels within engineering parenchymal tissues.

With reference to FIG. 2A, a schematic of the method 100 of producing a cellular support material 200 is provided. Each step of the method 100 will be described in detail below.

### Fabricating a Microfiber Lattice

In various aspects, the method 100 (FIG. 1) includes fabricating a microfiber lattice 202 at 110. In certain aspects, the microfiber lattice 202 is a temporary structure (i.e., a template material). In certain aspects, the microfiber lattice 202 is configured to be at least partially degraded after the associating or seeding with cells and encapsulating with a hydrogel matrix.

In various aspects, the microfiber lattice 202 includes a plurality of fibers 204. The fibers 204 may be disposed in a three-dimensional structure. The fibers may be oriented or disposed in any shape that is suitable to grow tissues (e.g., in any shape suitable to form a microvasculature). As will be discussed in greater detail herein, in certain aspects, the plurality of fibers are highly aligned to form a lattice structure (see, e.g., the microfiber lattice 202 of FIG. 2A). In certain aspects, the fibers 204 include a first portion of fibers 206 that are substantially aligned in a first direction A and a second portion of fibers 208 that are substantially aligned in a second direction B. In certain aspects, the second direction B is substantially orthogonal to the first direction A. In certain aspects, such as when the second direction B is substantially orthogonal to the first direction A, the first portion of fibers 206 and the second portion of fibers 208 connect or intersect at a plurality of intersection points 210 to form the 3D lattice structure.

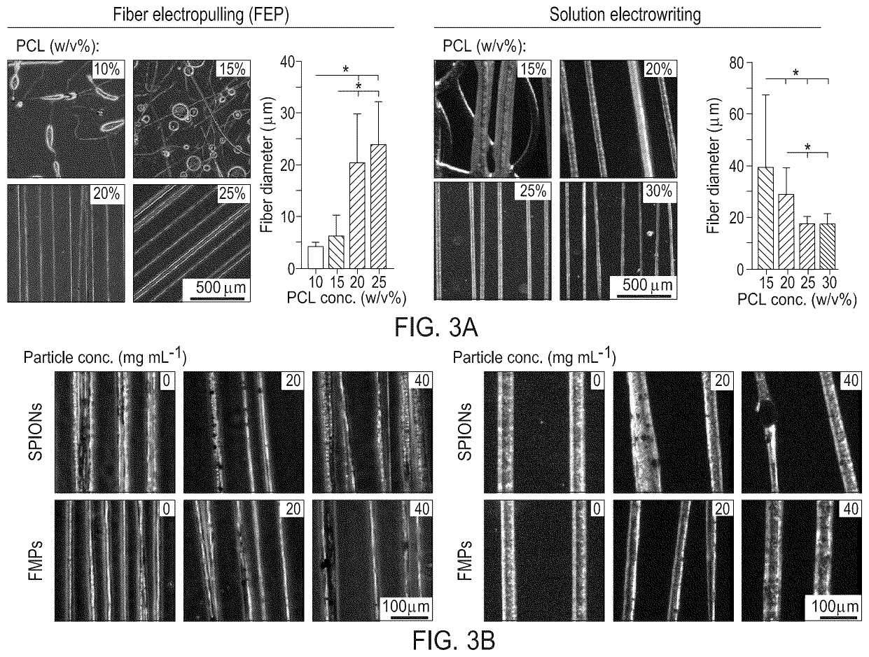

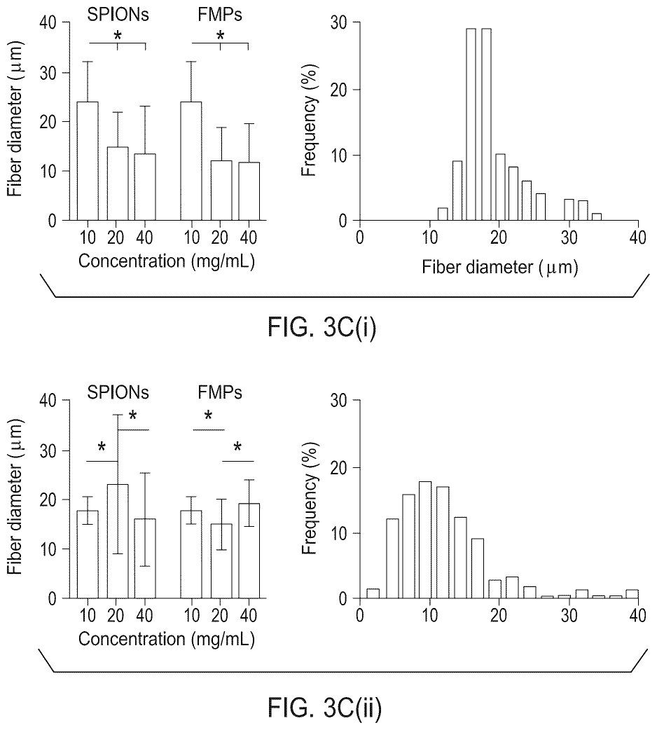

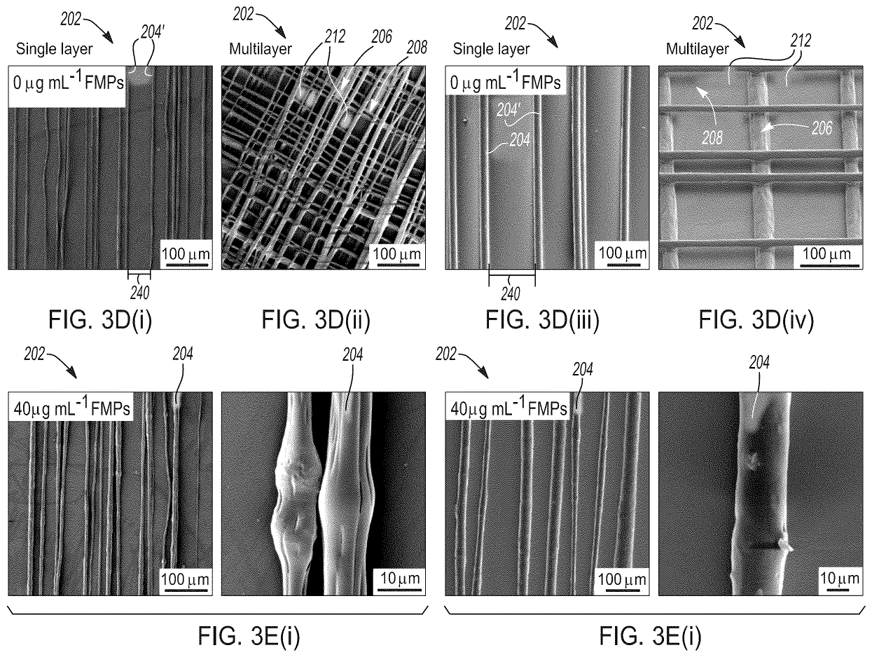

In certain aspects, the fibers 204 are oriented or spaced such that a plurality of voids 212 are distributed between the fibers. As best shown in FIGS. 2A and 3D, the voids 212 may be defined by the surfaces of the first portion of fibers 206 and the second portion of fibers 208. In certain aspects, the configuration of the fibers 204 forms voids 212 having an average volume sufficient to dispose a plurality of cells 220 therein.

In various aspects, the fibers 204 may include or be formed from a fiber precursor material 222. In certain aspects, the fiber precursor material 222 includes a porous polymer. In certain aspects, the fiber precursor material 222 includes a biodegradable polymer (i.e., the polymer may be configured to be selectively degraded by enzymes). In certain preferred aspects, the fiber precursor material 222 includes a polyester. By way of non-limiting example, the fiber precursor material 222 may include polycaprolactone (PCL).

In certain aspects, the fiber precursor material 222 includes a plurality of pores. At least a portion of the pores may define an average diameter that is capable of enclosing a magnetic particle therein. In certain aspects, the pores have an average diameter that is greater than or equal to about 5 nanometers (nm) to less than or equal to about 100 micrometers (μm). In certain aspects, as will be described in greater detail below, a superparamagnetic iron oxide nanoparticle (“SPION”) (having a diameter that is about 8 nm) or a ferromagnetic microparticle (“FMP”) (having a diameter that is about 5 μm) may be configured to be distributed in at least a portion of the pores.

In various aspects, each of the fibers 204 has a first dimension or diameter and a second dimension or length that are configured to simulate the size and structure of natural tissue. In certain variations, the microfiber lattice 202 may include fibers 204 having a uniform diameter. In certain other variations, the microfiber lattice 202 may include fibers 204 having a variety of different diameters. In certain aspects, the fibers 204 have an average diameter that is greater than or equal to about 5 micrometers (μm) to less than or equal to about 100 μm. More narrowly, the fibers 204 may have an average diameter that is greater than or equal to about 10 μm to less than or equal to about 50 μm. In certain aspects the fibers 204 have an average diameter that is greater than or equal to about 5 μm, optionally greater than or equal to about 10 μm, optionally greater than or equal to about 20 μm, optionally greater than or equal to about 30 μm, optionally greater than or equal to about 40 μm, optionally greater than or equal to about 50 μm, optionally greater than or equal to about 60 μm, optionally greater than or equal to about 70 μm, optionally greater than or equal to about 80 μm, or optionally greater than or equal to about 90 μm. In certain aspects, the fibers 204 have an average diameter than is less than or equal to about 100 μm, optionally less than or equal to about 90 μm, optionally less than or equal to about 80 μm, optionally less than or equal to about 70 μm, optionally less than or equal to about 60 μm, optionally less than or equal to about 50 μm, optionally less than or equal to about 40 μm, optionally less than or equal to about 30 μm, optionally less than or equal to about 20 μm, or optionally less than or equal to about 10 μm. The diameter and length of the fibers 204 may be tailored to achieve the desired fiber orientation and cell seeding characteristics of the cellular support material 200.

In various aspects, at least a portion of the plurality of fibers 204 includes at least one first magnetic particle 224 at least partially embedded therein. The first magnetic particle 224 may include a SPION, a FMP, or combinations thereof. As best shown in FIG. 3B, SPION and/or FMP particles may be readily incorporated into fibers with limited impact on microfiber deposition and resulting morphology.

In certain aspects, fabricating the microfiber lattice 202 includes magnetizing the microfiber lattice 202. In certain aspects, the first magnetic particles 224 are added or doped into the fiber precursor material 222 prior to fiber fabrication. In this regard, after fabricating the microfiber lattice 202, at least a portion of the fibers 204 have at least one magnetic particle 224 embedded therein. In certain aspects, the first magnetic particles 224 are included in the fiber precursor material 222 at a concentration of about 40 mg mL⁻¹. Above 40 mg mL⁻¹, magnetic particle concentration may be too high to consistently form fibers due to frequent interruptions in microfiber deposition.

Fabricating the microfiber lattice 202 may include solution electrowriting (SEW) (FIG. 2B) or fiber electropulling (FEP) (FIG. 2C). the fiber precursor material. In certain aspects, the microfiber lattice 202 is fabricated by SEW methods. SEW combines 3D printing with electrospinning at short spinneret to collector distances(e.g., via a grounded polymer solution extruder 226), such that electrostatic forces drive the formation and controlled deposition of polymeric microfibers 204 onto an oppositely charged collection surface 228 (i.e., a charged print stage).

In certain other aspects, the microfiber lattice 202 is fabricated by FEP methods. FEP involves the translation of a collection surface and resulting mechanical forces to pull polymeric microfibers from solution onto a rotating mandrel system 230. In the FEP method, electric fields are solely utilized to re-establish contact between the depositing microfiber and collection substrate should microfiber deposition be transiently disrupted.

In certain aspects, the spacing and the diameters of the fibers 204 are controlled using either SEW or FEP methods by tuning the concentration and resulting viscosity of the polymer solutions in the fiber precursor material 222. As shown in FIG. 3A, in one example, forming a microfiber lattice using FEP methods and a fiber precursor material including about 10 to about 15 w/v % PCL results in inconsistent microfiber formation and highly variable fiber diameter. In another example, however, a fiber precursor material including higher concentrations of 20 and 25 w/v % PCL yields uniform fiber diameters and more consistent deposition. Generally, the fiber diameter increases with increasing PCL concentrations when solutions are processed by FEP but interestingly, the opposite relationship is noted when identical solutions are processed into microfibers using SEW. Since FEP is mechanically driven, increasingly viscous solutions correspond to larger fiber diameters due to the constant force applied to the solution at a given collection speed. In contrast, during SEW microfiber deposition, the force applied to the polymer solution and ensuing thinning of the drawn material is a function of charge density which increases with polymer concentration.

In one example, as best shown by FIG. 3C, by doping the fiber precursor material 222 with FMPs (i.e., the first magnetic particle 224 comprises a FMP), the resulting fiber diameters decreases when processed into microfibers by FEP (FIG. 3C(i)) but not by SEW (FIG. 3C(ii). FMP-doped fibers produced from a 25 w/v % PCL solution are smaller when generated by FEP (11.87 μm+/−7.67 μm) (FIG. 3C(i)) as compared to SEW (19.07 μm+/−4.71 μm) (FIG. 3C(ii)). This decrease in fiber diameter specifically with FEP is likely due to the interruption of polymer extrusion caused by the presence of the particles 224. Distinct from SPIONs, the innate magnetic poles within each FMP can be reoriented by briefly placing the particles in a high strength magnet. SPIONs, in contrast, are only transiently magnetized when in the presence of a magnetic field. Due to the potential to permanently reorient the magnetic poles of FMPs, the highest concentration of FMPs (40 mg mL⁻¹) is used in all subsequent studies.

Both SEW and FEP methods fabricate lattices 202 composed of multi-layered microfibers 204 to generate user-defined capillary architectures. For example, FIGS. 3D(i)-(iv) show scanning electron microscope (SEM) images of FEP single (FIG. 3D(i)) FEP multi-layer (FIG. 3D(ii)), SEW single (FIG. 3D(iii)) and SEW multi-layer (FIG. 3D(iv)) PCL (25 w/v %) lattices.

While both SEW and FEP methods are capable of fabricating multi-layer microfiber lattices 202, in certain aspects, due to the 3D printing nature of SEW (as compared to FEP which requires manual reorientation of the collection substrate), this method could be utilized in the future to create scalable, customized multi-layer lattices of desired geometries for specific tissues of interest. Further, As shown in FIGS. 3E(i) (FEP fabricated) and 3E(ii) (SEW fabricated), SEM images show that FMP-doped microfibers 204 do not have significantly different morphology from non-loaded microfibers 204′ (see, e.g., non-loaded microfibers 204′ of FIG. 3D(i)-(iv)).

In various aspects, in fabricating the microfiber lattice 202, the spacing between microfibers 204 (i.e., the dimension and/or volume of the voids 212) may be controlled. In certain aspects, increasing the fiber diameter may decrease a dimension 240 (e.g., the space) between each of the fibers 204. Decreasing a dimension between each of the fibers 204 may consequently decrease the dimension and/or volume of the voids 212. Conversely, in certain aspects, decreasing the fiber diameter may increase the dimension 240 between each of the fibers 204, thereby increasing the dimension and/or volume of the voids 212.

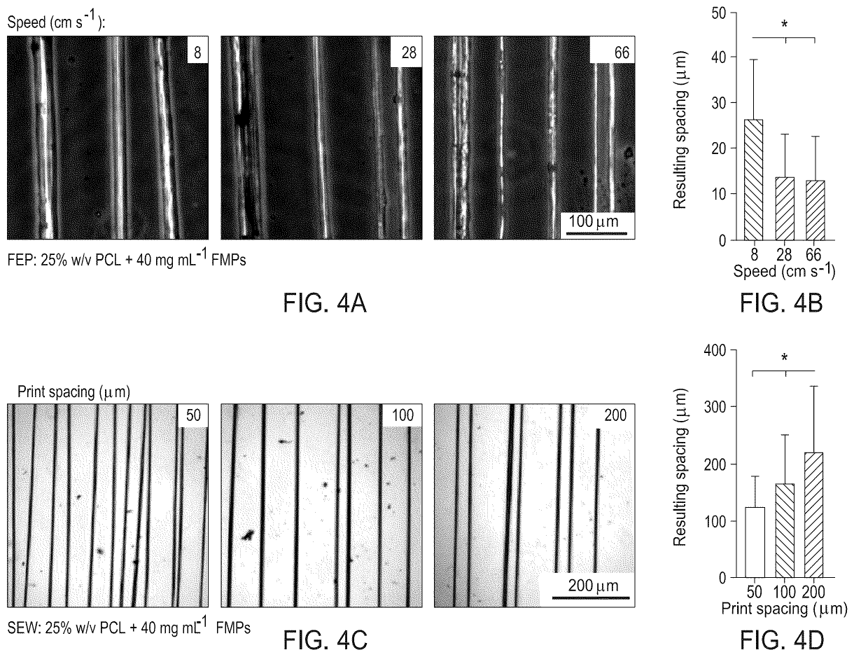

With reference to FIGS. 4A-4B, in certain aspects, utilizing FEP, fiber diameter is controlled via the linear rotation speed of the collecting mandrel 230 (FIG. 2C). In certain aspects, the linear rotation speed of the FEP method is greater than or equal to 5 cm s⁻¹ to less than or equal to about 66 cm s⁻¹. In one example, using a fiber precursor material including 25 w/v % PCL, the slowest rotation speed enabling constant fiber deposition (8 cm s⁻¹) yielded deposited fibers at about 25 μm diameter. Increasing mandrel rotation speed may decrease the average fiber diameter to about 15 μm. However, rotation speeds that are higher than 66 cm s⁻¹ may prevent consistent fiber deposition due to fiber breaking from mismatched polymer extrusion and deposition rates. For example, as shown in FIG. 4A, transmitted light images of FEP produced 25 w/v % PCL+40 mg ml⁻¹ FMPs fiber diameters are shown at rotation speeds of 8 cm s⁻¹, 28 cm s⁻¹, and 66 cm s⁻¹. The corresponding diameter dimensions (in micrometers (μm)) are shown in FIG. 4B. The fiber diameter at a speed of 8 cm s⁻¹ was larger than the fiber diameters at speeds of 28 cm s⁻¹ and 66 cm s⁻¹.

With reference to FIGS. 4C-4D, in certain other aspects, utilizing SEW methods, the travel path of the 3D printer nozzle may be encoded to space fibers at defined distances apart. FIG. 4C shows transmitted light images of 25 w/v % PCL+40 mg ml⁻¹ FMPs fiber diameters produced by SEW at 50 μm, 100 μm, and 200 μm preset fiber distances. In certain aspects, as shown in FIG. 4D, increasing fiber spacing is commensurate with increasing coded printing space. In one example, however, the actual spacing between fibers is about 75 μm greater than the intended distance, likely due to variations in the electrostatic field. In certain aspects, inconsistencies in electric field may arise from the deposition of nearby deposited fibers which contribute static charge buildup and repel subsequently deposited fibers.

### Introducing Cells to the Microfiber Lattice

With renewed reference to FIG. 2A, in various aspects, the method 100 (FIG. 1) further includes introducing cells 220 to the microfiber lattice 202 at 120 (FIG. 1). In certain aspects, the method includes introducing a suspension of cells 220 to the microfiber lattice 202. The cells 220 may include any cell that is capable of engulfing and/or ingesting a magnetic particle by endocytosis. The cells 220 may be selected to meet the particular function and characteristics of the engineered tissue. In certain aspects, the cells 220 include parenchymal cells, such as when the cellular support material 200 is used to form parenchymal tissue. By way of non-limiting example, in certain aspects, the cells 220 may include endothelial cells (ECs).

The highly porous nature of lattices composed of microfibers generally presents a challenge for cell seeding, such as EC seeding, as ECs in suspension rapidly settle prior to lattice attachment resulting in non-uniform seeding. In various aspects of the present disclosure, at least a portion of the cells 220 include at least one second magnetic particle 244 at least partially embedded therein. In certain aspects, the second magnetic particle 244 may include a SPION, a FMP, or combinations thereof. In certain aspects, the first magnetic particle 224 of the microfiber lattice and the second magnetic particle 244 of the cells are independently selected from the group consisting of: a SPION, a FMP, or combinations thereof. In one example, the first magnetic particle 224 includes a FMP, the cells 220 include ECs, and the second magnetic particle 244 includes a SPION. By loading the cells 220 with magnetic particles, cell 220 attachment to the magnetized microfiber lattices 202 is enhanced.

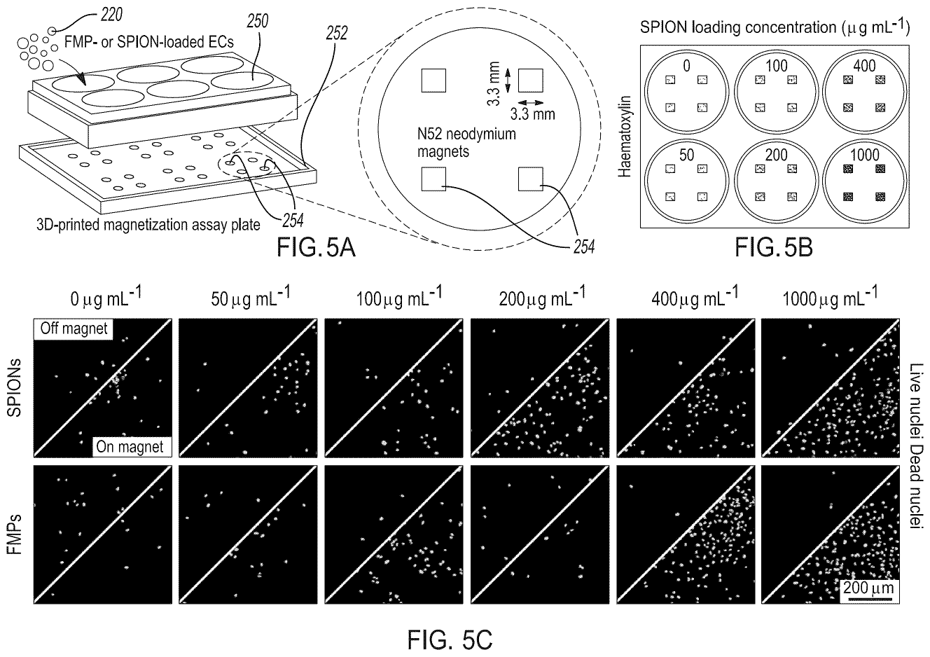

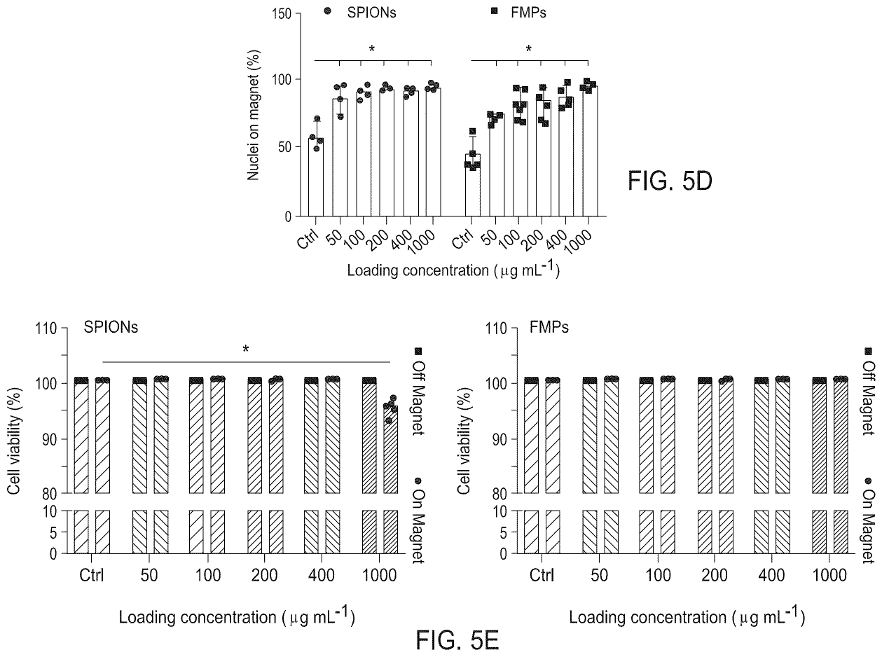

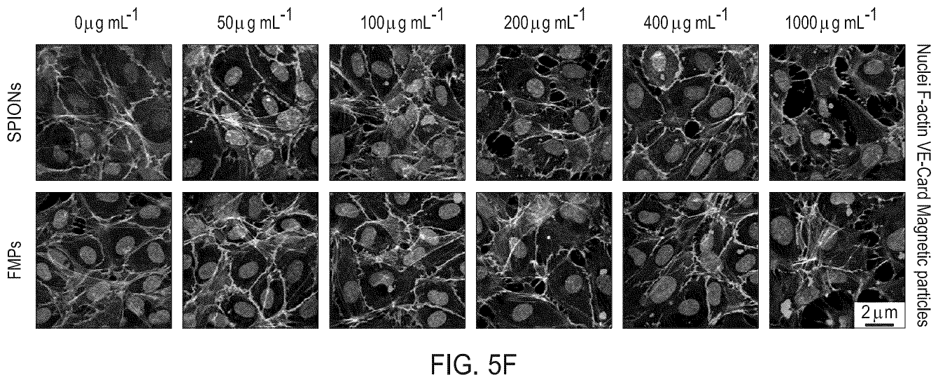

In various aspects, loading the cells 244 with magnetic particles 244 enables cell magnetization with limited impact on cell viability and barrier function. In certain aspects, the cells 220 are loaded with magnetic particles 244 via endocytosis. In certain aspects, prior and/or concurrently to the introducing, the cells 220 may be exposed to a concentration of magnetic particles 244 that is greater than or equal to about 400 μg mL⁻¹ to less than or equal to about 1000 μg mL⁻¹. With reference to FIG. 5A-5F, in one example, ECs are fed SPIONs and/or FMPs at various concentrations diluted in cell culture media and cultured overnight to allow for particle uptake via endocytosis. To assess the degree of cell magnetization, EC suspensions are plated onto tissue culture plastic (TCP) well-plates 250 positioned over a 3D printed plate holder 252 containing N52 neodymium magnets 254 (FIG. 5A). The percentage of cells adhering above magnets is interpreted as a measure of cell magnetization efficiency, where equal distribution of cells on magnet and non-magnet positions results in a magnetization efficiency of 50% (FIGS. 5B-5C). As best shown in FIG. 5D, all particle concentrations increase the percent of cells seeded over magnets.

In certain aspects, loading the cells 220 with magnetic particles 244 does not impact the viability of the cells 220. With reference to FIGS. 5C-5F, cell viability is analyzed by propidium iodide staining (FIG. 5C). Notably, only a concentration of 1000 μg mL⁻¹ SPIONs resulted in a significantly lower cell viability than control cells that were not fed magnetic particles (FIG. 5E). Despite magnetic particle loading with either SPIONs or FMPs, ECs retained their ability to localize VE-Cadherin to adherens junctions, the cell-cell adhesion critical to endothelial barrier function (FIG. 5F).

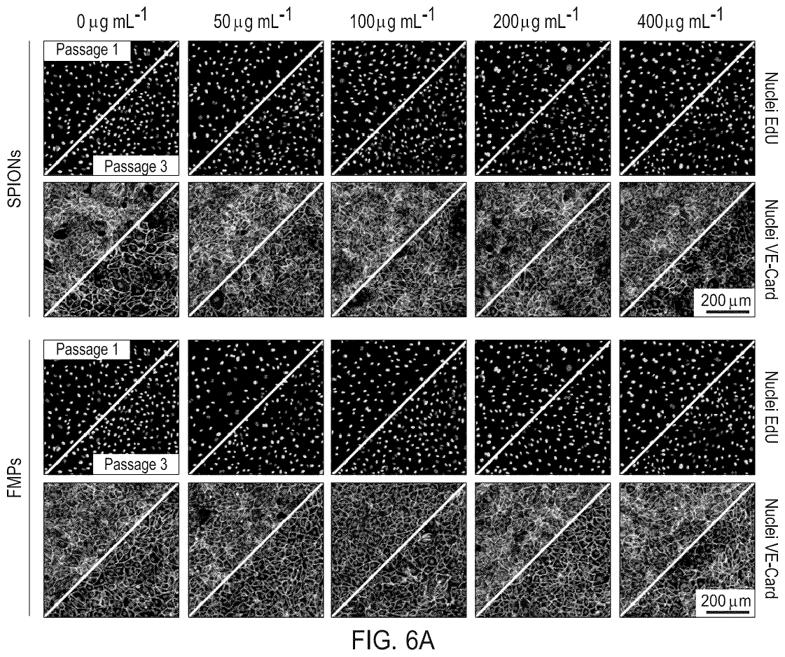

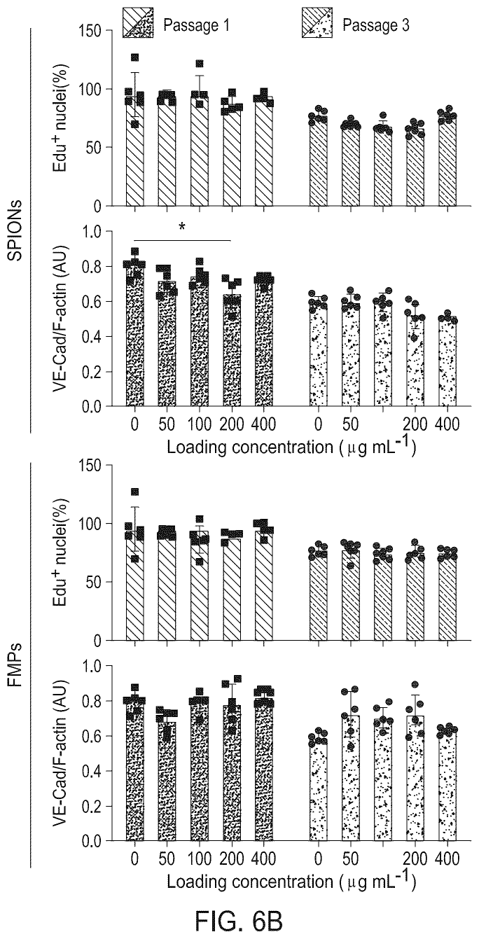

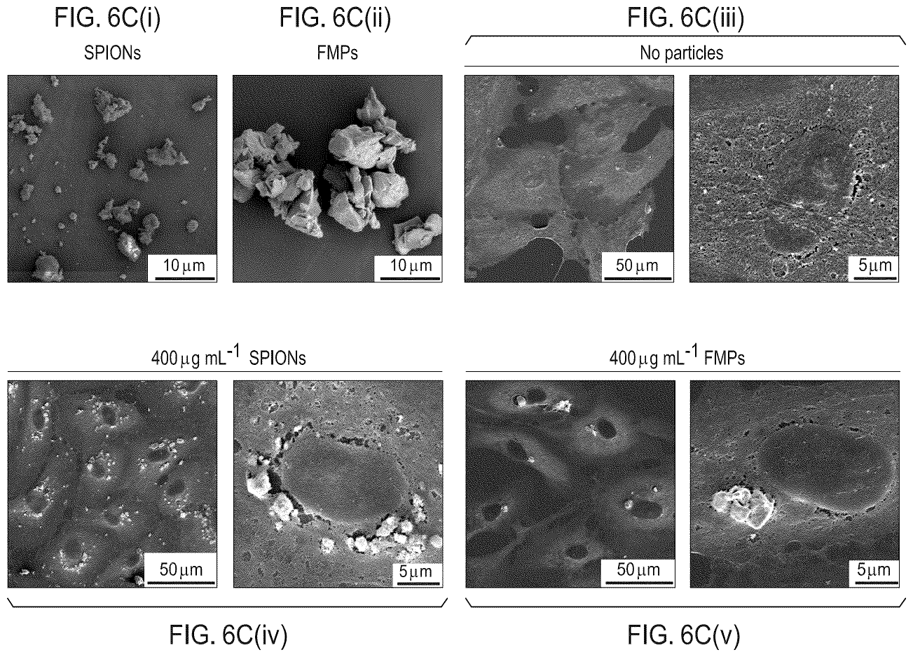

In certain aspects, loading the cells 220 with magnetic particles 244 does not alter long-term cell function. With reference to FIGS. 6A-6C, ECs are passaged up to three times after magnetic particle loading, and at the first and third passage are assessed for proliferative capacity and ability to robustly form adherens junctions (FIG. 6A). Maximal concentration of 400 μg mL⁻¹ SPIONs are tested in these studies due to noted decreases in cell viability at 1000 μg mL⁻¹ (FIG. 5E). For both particle types across all tested concentrations, proliferation is unaffected as quantified by EdU assay (FIG. 6B). Additionally, ECs are able to form comparable adherens junctions across all conditions, suggesting retained capacity to form robust cell-cell adhesions required for endothelial barrier function (FIG. 6B). Endocytosis of SPIONs appears more efficient than FMPs, as shown in representative SEM images (FIG. 6C). Notably, both SPIONs and FMPs accumulate around the nucleus of each of the cells. Due to the difference in particle sizes between SPIONs (about 8 nm in diameter) and FMPs (about 5 μm in diameter), the smaller SPIONs, which should not form aggregates prior to endocytosis, may be more easily endocytosed than FMPs. Based on these results, 400 μg mL⁻¹ SPIONs is utilized in subsequent studies given their more homogeneous distribution and undetectable impact on EC viability compared to FMPs.

With renewed reference to FIG. 2A, in various aspects, the method 100 includes introducing the cells 220 to the microfiber lattice 202 after at least a portion of the microfiber lattice 202 and a portion of the cells 220 include at least one first and one second magnetic particles 224, 244, respectively. In certain aspects, the introducing includes incubating the magnetized microfiber lattice 202 in a suspension of magnetized cells 220.

### Associating the Cells With the Plurality of Fibers in the Microfiber Lattice

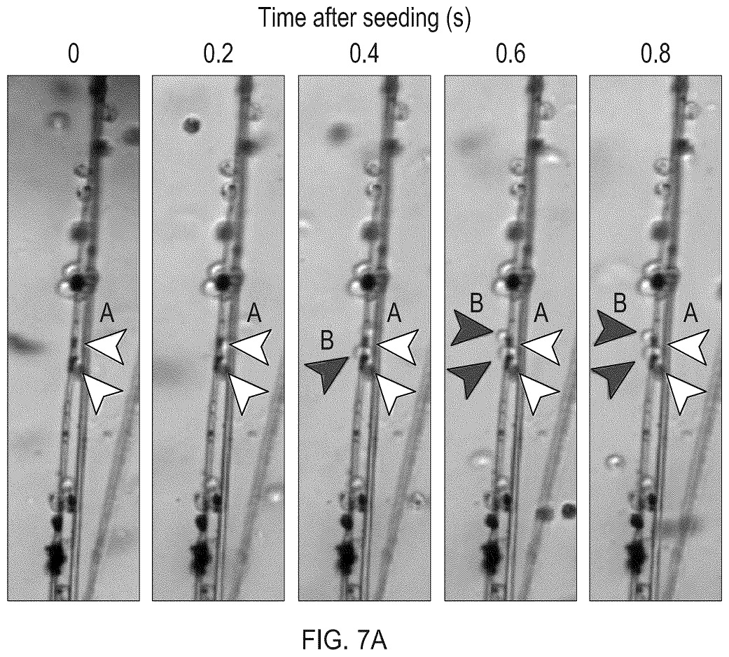

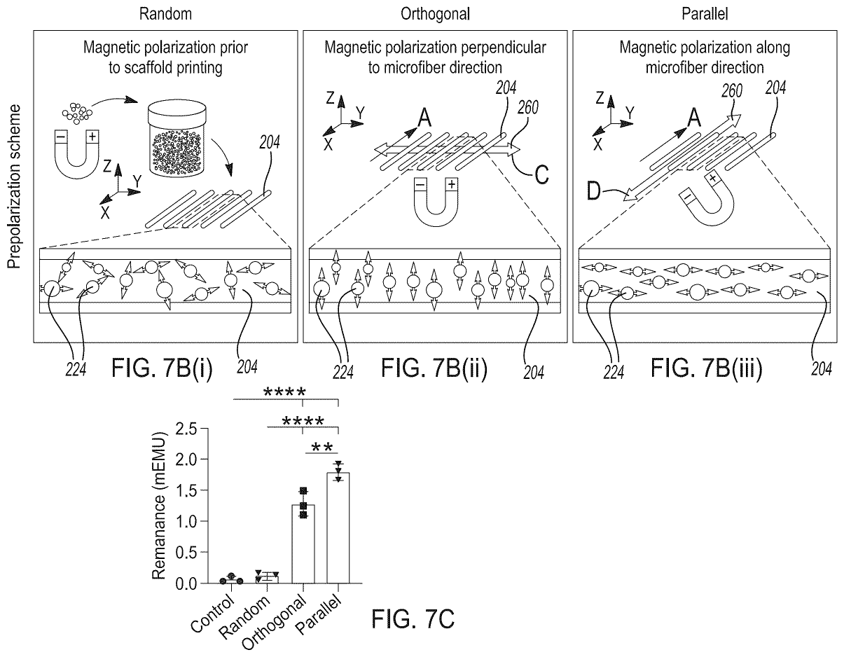

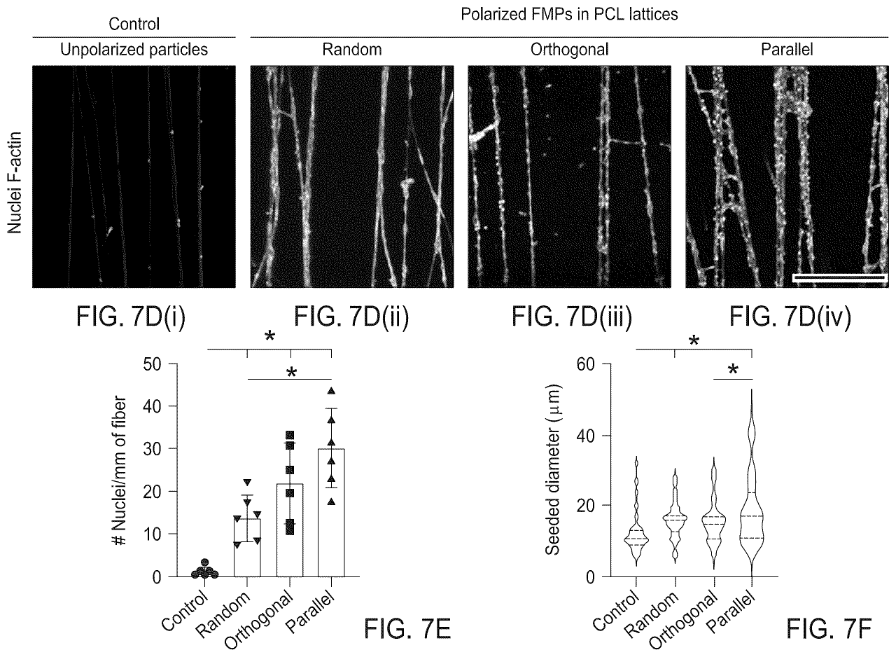

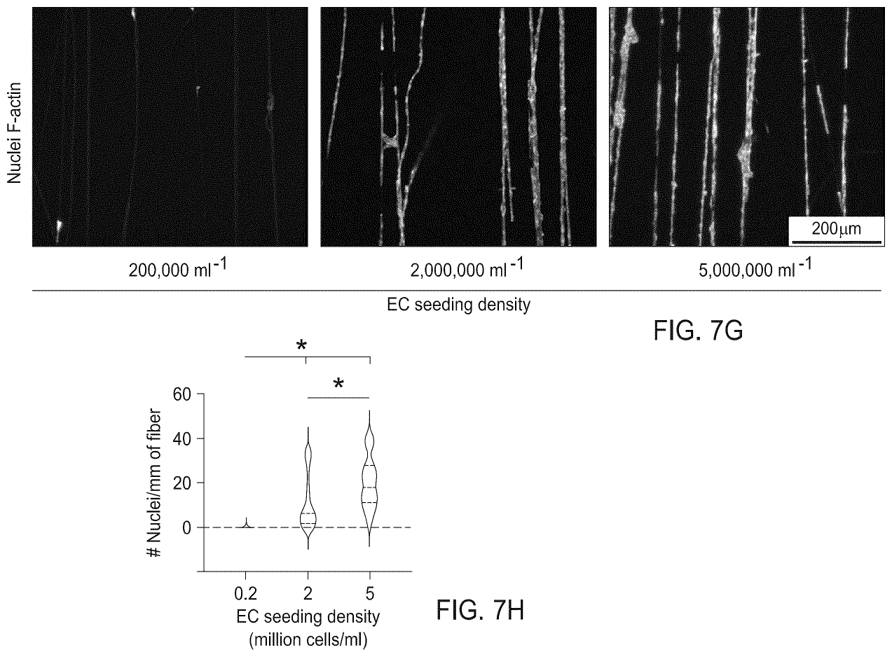

In various aspects, the method 100 (FIG. 1) further includes associating the cells 220 with the plurality of fibers 204 in the microfiber lattice 220 at 130 (FIG. 1). As will be discussed in greater detail below, in certain aspects, the associating includes applying a magnetic field 260 (FIG. 7B(ii)-(iii)) to the microfiber lattice 202 having the suspension of cells 220 therein (e.g., when the first magnetic particles 224 include SPION particles). In certain other aspects, prior to the associating, the first magnetic particles 224 of microfiber lattice 202 are polarized (FIG. 7B(i)) (e.g., when the first magnetic particles 224 include FMP particles). In other words, the microfiber lattice 202 including the first magnetic particles 224 embedded therein may be pre-polarized prior to associating or seeding the cells to the microfiber lattice.

FMPs innately contain small internal magnetic domains with randomly aligned magnetic dipoles. This random alignment of the magnetic domains within a particle cancels out the resulting net magnetic field, effectively resulting in an “unmagnetized” state. However, the magnetic domains in FMPs can be rewritten by applying a strong external magnetic field to saturation, thereby aligning each domain's magnetic pole to strengthen the net particle magnetic field. When the strong external field is removed, the now polarized particles preserve the co-aligned pole and magnetic field (high remanence) (FIG. 7C). In contrast, superparamagnetic particles such as SPIONs only temporarily magnetize under a stronger magnetic field and will return to their non-magnetic state upon removal of the external magnetic field (low remanence). For this reason, a high concentration 40 mg mL⁻¹ FMPs solution is used for lattice magnetization with the thought that magnetically polarized FMPs (either prior to or after lattice fabrication) would enhance microfiber magnetization and attract SPION-carrying ECs during seeding.

As shown in FIG. 7A, the magnetic field produced by lattice-embedded FMPs is sufficiently strong to attract SPION-loaded ECs and promote rapid, selective attachment during seeding with magnetized cell suspensions. In certain aspects, pre-polarizing the first magnetic particles 224 further enhances the efficiency of associating or seeding of the cells 220 to the fibers 204.

In certain aspects, as shown in FIG. 7B(i), the first magnetic particles 224 are polarized prior to fabricating the microfiber lattice 202. As shown in FIG. 7D(ii), in certain aspects, when the magnetic particles 224 are polarized prior to fabricating the microfiber lattice 202, the magnetic fields of each of the fibers lack alignment (i.e., form “random” scaffolds).

In certain other aspects, as shown in FIGS. 7B(ii)-(iii), the first magnetic particles 224 are polarized after lattice fabrication. Polarization after the fabrication of the microfiber lattice 202 may include applying a magnetic field 260 to the microfiber lattice 202 to magnetize the FMP particles. In certain aspects, at least a portion of the fibers 204 (e.g., first portion 206) are substantially aligned along the first direction A and the polarizing the microfiber lattice 202 includes applying a magnetic field 260 to the microfiber lattice in a third direction C substantially orthogonal to the first direction A (FIG. 7B(ii)). In certain other aspects, at least a portion of the fibers (e.g., first portion 206) are substantially aligned along the first direction A and the polarizing the microfiber lattice 202 includes applying a magnetic field 260 to the microfiber lattice in a fourth direction D substantially parallel to the first direction A (FIG. 7B(iii)). In certain aspects, the magnetic field 260 has a magnetic flux density that is about 1 T.

As shown in FIG. 7C, the resulting remanence (magnetization at zero field) of the microfibers 204 in a vibrating-sample magnetometer varies by magnetic microdomain. Control, non-magnetized scaffolds exhibit very low remanence since magnetic particles are not polarized. ‘Random’ scaffolds contain prepolarized FMPs (e.g., as shown in FIG. 7B(i)) that lacked alignment after microfiber printing. Due to the random alignment of FMPs with respect to each other, the net magnetization at zero field is low. However, orthogonal (e.g., 7B(ii)) and parallel (e.g., FIG. 7B(iii)) samples exhibit a non-zero remanence since they were polarized after microfiber fabrication in uniform directions. Applying the magnetic field 260 in Direction D that is parallel to the first direction A fiber alignment exhibits the highest remanence of the sample set and provides more homogeneous magnetization and field lines around the diameter of the fiber 204. Applying the magnetic field 260 in Direction C that is orthogonal to the first direction A fiber alignment results in anisotropic fields, which may be less favorable for homogeneous cell seeding and coverage of the fibers 204. As shown in FIG. 7D(i), without pre-polarizing FMP microdomains, SPION-loaded ECs fail to attach to the lattice due to weak FMP-generated magnetic fields and low remanence, in agreement with FIG. 7C.

In certain aspects, all three pre-polarization techniques (i.e., random (FIGS. 7B(i) and 7D(ii)), orthogonal (FIGS. 7B(ii) and 7D(iii)), and parallel (FIGS. 7B(iii) and 7D(iv)) to associate or seed cells 220 to the microfiber lattice 202 seed the cells 220 more efficiently than nonpolarized, low-remanence controls (FIG. 7D(i)). Parallel FMP polarization seeds at the highest efficiency, while pre-polarized FMPs with putatively random orientation with PCL microfibers proves less efficient, as measured by the density of attached cells (FIG. 7E). Upon closer analysis, the morphology of cells attached to orthogonal or parallel polarized lattice appear more rounded and less spread, perhaps due to an excessive density of attached cells that subsequently prevented cell spreading (FIG. 7D). The density of attached ECs correlates with the resulting diameter of cell-seeding PCL microfibers (FIG. 7F). Further, the concentration of cells in suspension correlates with the degree of attachment, as expected. A seeding density of 2,000,000 cells mL⁻¹ results in complete cellular coverage of PCL microfibers while allowing for EC spreading along microfibers (FIGS. 7G-H). Given the efficient seeding, resulting capillary-scale diameters, and uniform distribution of ECs on PCL microfibers containing pre-polarized FMPs, 2,000,000 cell mL⁻¹ seeding density and parallel FMP pre-polarization is employed in all subsequent experiments.

### Encapsulating the Microfiber Lattice and Associated Cells With a Hydrogel

Referring back to FIG. 2A, in various aspects, the method 100 (FIG. 1) further includes encapsulating the microfiber lattice 202 and associated cells 220 with a hydrogel 270 at 140 (FIG. 1). In certain aspects, the hydrogel 270 includes or is formed from a polysaccharide, a polypeptide (e.g., collagen, fibronectin, gelatin, derivatives thereof, and combinations thereof), a synthetic polymer, or combinations thereof. In certain aspects, the hydrogel 270 fully encapsulates the microfiber lattice 202 and associated cells 220, such that the cells 220 and the microfiber lattice 202 are disposed within surfaces 282 defined by the hydrogel.

### Removing at Least a Portion of the Plurality of Fibers

In various aspects, the method 100 (FIG. 1) further includes removing at least a portion of the microfiber lattice 202 at 150 (FIG. 1). In certain aspects, removing at least a portion of the microfiber latter 202 includes removing or degrading at least a portion of the fibers 204. In certain aspects, the entire microfiber lattice 202 is removed. In certain aspects, the removal of fibers 204 may be triggered after the complete seeding of cells 220 onto the microfiber lattice 202. Pre-mature removal of fibers 204 could hinder the growth of tissue on the cellular support material 200.

In certain aspects, the removing includes degrading the portion of the microfiber lattice 202. Any suitable method to selectively degrade the polymer fibers 204 while maintaining cell 220 viability may be used. In certain aspects, the degrading includes enzymatically degrading at least a portion of the fibers 204 via an enzyme 272. In certain aspects, the enzyme 272 may include lipase utilized to enzymatically degrade the fibers. In certain other aspects, non-enzymatic methods such as light-induced degradation may selectively degrade the microfiber lattice 202 without harming the viability or function of the cells 220.

By way of non-limiting example, to selectively remove PCL microfibers 204 after endothelialization and encapsulation in the hydrogel 270 of choice along with desired parenchymal cells, a bacterial lipase that efficiently and selectively cleaves ester bonds within the PCL backbone is used.

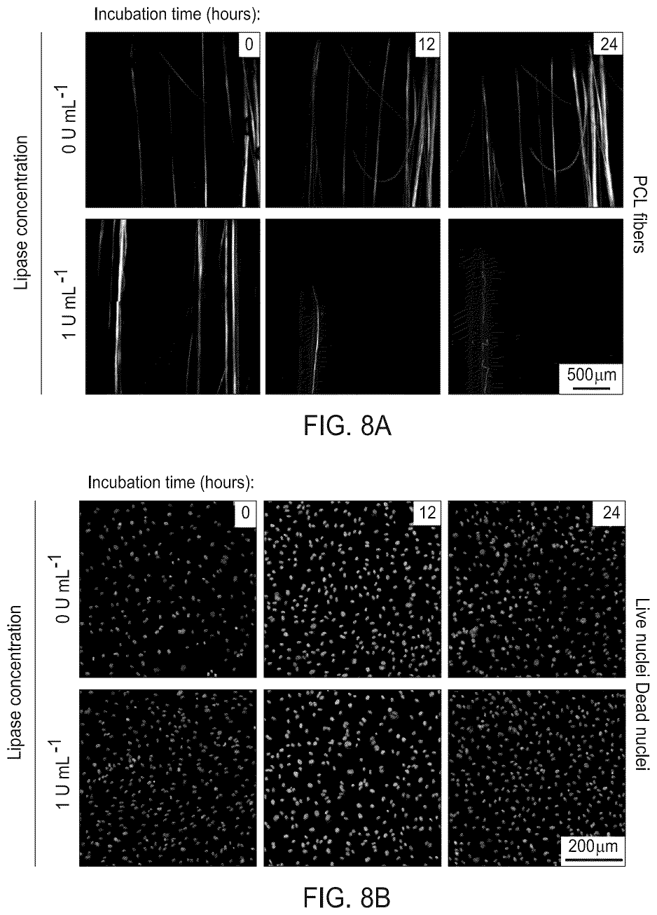

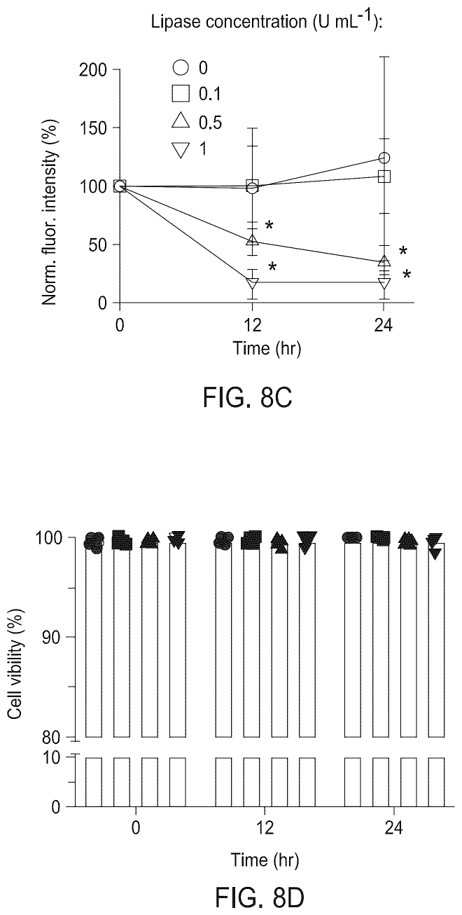

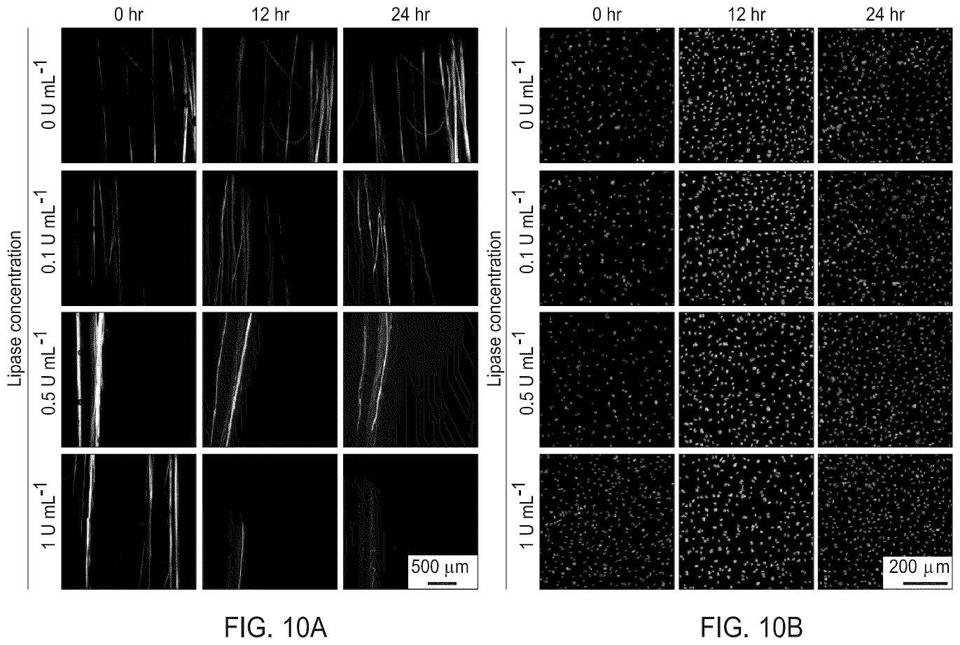

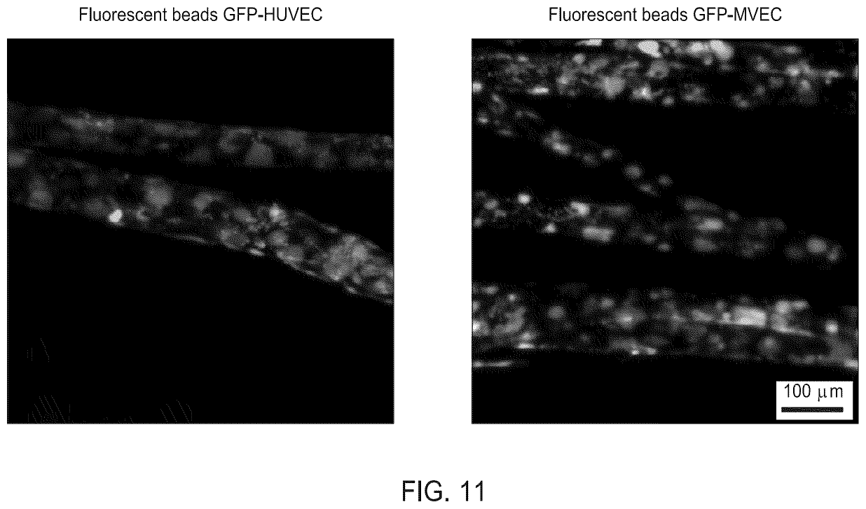

In certain aspects, the rate of degradation may increase with an increased concentration of enzyme 272. With reference to FIG. 8, fluorophore-doped PCL microfibers are treated with varying concentrations of Pseudomonas sp. lipase and imaged over 24 hours (FIGS. 8A and 10A-B). Exposure to bacterial lipase degraded PCL microfibers as a function of lipase concentration, where the highest concentration (1 U mL⁻¹) completely degraded microfibers within 12 hours (FIG. 8C). Importantly, exposure of ECs to even the highest concentration of lipase tested did not negatively impact EC viability (FIGS. 8B and 8D). Based on these studies, 1 U mL⁻¹ lipase for 24 hours is determined to be sufficient for PCL microfiber degradation and is used in all subsequent cell studies.

In various aspects, degrading at least a portion of the microfiber lattice 202 forms a plurality of channels 280. In certain aspects, the plurality of channels 280 are microchannels. In certain aspects, the channels 280 are disposed in the direction of the degraded fiber. For example, when the degraded fiber was included in the first portion of fibers 206 oriented in direction A, the channels 280 are disposed in direction A. Additionally or alternately, when the degraded fiber was included in the second portion of fibers 208 oriented in direction B, the channels 280 are disposed in direction B. In other words, the plurality of channels 280 includes a first portion 290 of the channels 280 aligned in the first direction A and a second portion 292 of the channels 280 aligned in the second direction B. In certain aspects, the channels 280 are defined by the surfaces 282 of the hydrogel. The cells 220 may be embedded within the surfaces defined by the channels.

An average diameter 284 of the channels may be tailored by the fiber diameter, the cell diameter, and the compression of the hydrogel after encapsulation. In certain aspects, the channels 282 have an average diameter 284 that is greater than or equal to about 5 μm to less than or equal to about 100 μm. More narrowly, the channels 282 may have an average diameter 284 that is greater than or equal to about 10 μm to less than or equal to about 50 μm. In certain aspects, the channels 282 have an average diameter 284 that is greater than or equal to about 5 μm, optionally greater than or equal to about 10 μm, optionally greater than or equal to about 20 μm, optionally greater than or equal to about 30 μm, optionally greater than or equal to about 40 μm, optionally greater than or equal to about 50 μm, optionally greater than or equal to about 60 μm, optionally greater than or equal to about 70 μm, optionally greater than or equal to about 80 μm, or optionally greater than or equal to about 90 μm. In certain aspects, the channels 282 have an average diameter 284 that is less than or equal to about 100 μm, optionally less than or equal to about 90 μm, optionally less than or equal to about 80 μm, optionally less than or equal to about 70 μm, optionally less than or equal to about 60 μm, optionally less than or equal to about 50 μm, optionally less than or equal to about 40 μm, optionally less than or equal to about 30 μm, or optionally less than or equal to about 20 μm.

### Cellular Support Material Characterization

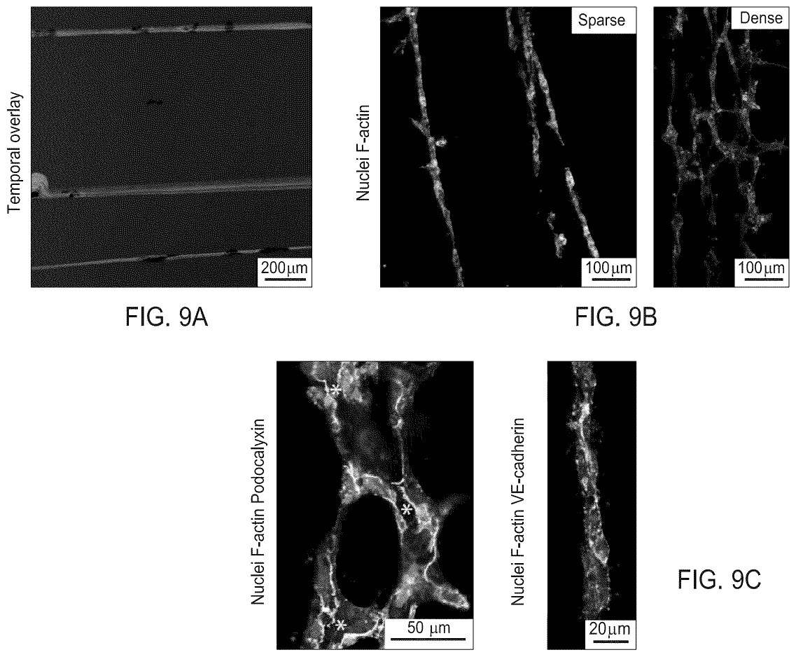

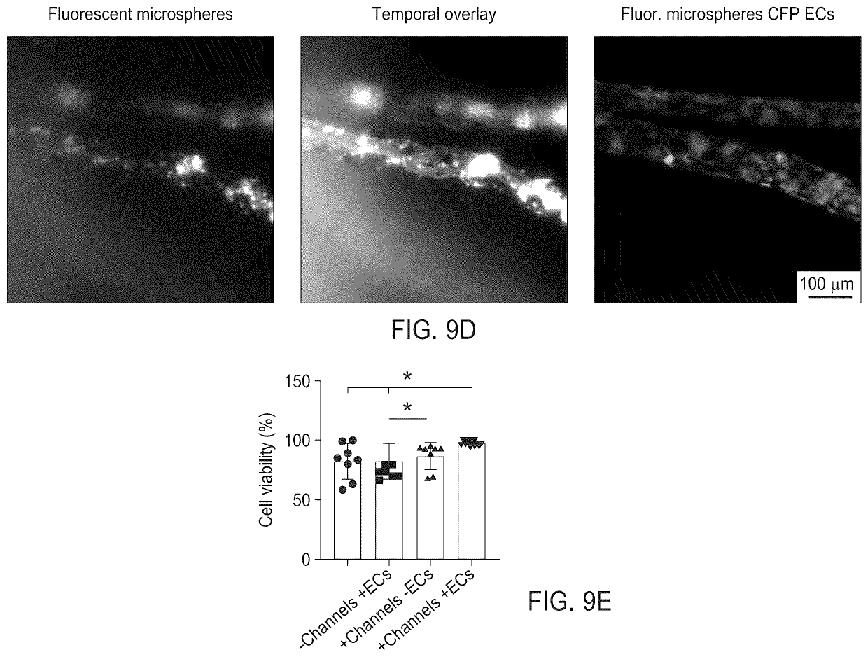

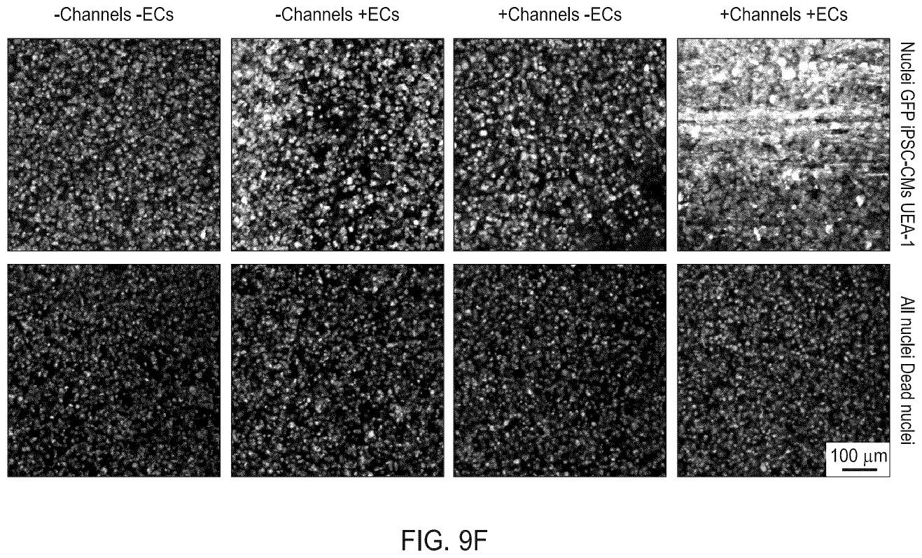

In various aspects, the present disclosure provides a cellular support material 200 that is capable of growing cells thereon. With reference to FIG. 9A-9F, fluorescent microsphere perfusion demonstrated fluid flow through the templated microchannels (FIG. 9A). To assess microvessel assembly, EC spreading and morphology are investigated after 2 days of culture on PCL lattices encapsulated within a type I collagen hydrogel. Collagen is cast around the lattices followed by lipase degradation of PCL. ECs maintained their spread morphology reflecting the original PCL lattice design (FIG. 9B). Interestingly, occasional EC extension and anastomosis between adjacent, parallel microvessels is observed when microvessels are positioned less than or equal to about 100 μm apart (FIG. 9B). Microvessels express VE-cadherin at cell-cell junctions and demonstrate proper apical-basal polarity and lumenization, as visualized by the apical/lumenal marker podocalyxin (FIG. 9C). Lumenization is further validated by fluorescent bead perfusion of acellular 4 mg mL⁻¹ collagen hydrogels and GFP HUVEC and GFP MVEC lined vessels in 10 mg mL⁻¹ fibrin hydrogels (FIGS. 9C-D and 11).

To test whether magnetically assisted capillary templating and coculture with ECs could support the high metabolic demands of cardiomyocytes, induced pluripotent stem cell-derived cardiomyocytes (iPSC-CMs) are incorporated within 10 mg mL⁻¹ fibrin gels at high density (40,000,000 cell mL⁻¹). After 5 days in culture following PCL degradation, cell viability is assessed by staining for Phospho-Histone H2A.X, which is a marker of DNA damage. Hydrogels lacking predefined channels, regardless of EC presence, result in low iPSC-CM viability (FIGS. 9E-F). The inclusion of acellular microchannels or alternatively admixed ECs lead to an increase in iPSC-CM viability. Interestingly, highest iPSC-CM viability occurs in tissue with endothelialized microchannels. These findings suggest the importance of not only channels for nutrient delivery but also endothelial cells which reflects current literature concerning the relationship between endothelial cells and cardiomyocytes. Specifically, ECs are known to secrete multiple paracrine signals, such as nitric oxide, neuregulin-1, prostaglandin E2, prostacyclin, and parathyroid hormone-related peptide that have previously been reported to support adult cardiac tissue function. Nitric oxide is primarily produced by ECs and has been shown to support CM contractility. ECs also secrete numerous proteins and growth factors, such as parathyroid hormone-related peptide, to modulate CM structure and function. Although these secretory factors are not independently investigated in this study, results suggest that the presence of endothelial cells on the channels play an important role in cardiomyocyte function that may influence cell processes and viability.

Further, UEA-1 positive ECs largely remained localized to the channels created by the PCL networks (FIG. 9F). As HUVECs do not readily develop vasculature in high density fibrin hydrogels via vasculogenic self-assembly, the results suggest magnetic patterning of endothelial structures can facilitate vascularization in high density hydrogels. Using this approach, inclusion of EC-lined channels in cardiac tissues improves tissue survival and enables fabrication of larger cardiac tissue models. In certain aspects, manipulating the design and configuration of the microfiber lattice could additionally impact the organization and anisotropy of the parenchymal tissue.

As such, the present disclosure provides the cellular support material 200. The cellular support material 200 includes the plurality of microchannels 280 defined in the hydrogel matrix 270. The microchannels 280 may define the plurality of surfaces 282. The first portion 290 of the plurality of microchannels 280 are aligned in the first direction A. In certain aspects, the second portion 292 of the plurality of microchannels 280 are aligned in the second direction B. As shown in FIG. 2A, the second direction B may be oriented substantially orthogonally to the first direction A. The cellular support material 200 further includes the plurality of cells 220 embedded within the surfaces 282 defined by the plurality of microchannels 280. In certain aspects, the cells 220 include at least one magnetic particle 244 (e.g., a SPION and/or a FMP) at least partially embedded therein.

In certain aspects the microchannels 280 have an average diameter 284 that is greater than or equal to about 5 μm to less than or equal to about 100 μm.

In certain aspects, the hydrogel matrix 270 includes or is formed from a polysaccharide, a polypeptide (e.g., collagen, fibronectin, gelatin, derivatives thereof, and combinations thereof), a synthetic polymer, or combinations thereof.

In certain aspects, the cells 220 include ECs. In certain aspects, the at least one magnetic particle 244 includes a SPION.

In various aspects, the cellular support material 200 may be utilized to grow cells. In certain aspects, a method of forming a microvasculature may include growing cells on the cellular support material until the microvasculature is formed.

Moreover, the present disclosure provides a template for forming a cellular support material. The template includes the plurality of microfibers 204 defining the three-dimensional lattice 202 and including the plurality of voids 212 between the respective microfibers 204. In certain aspects, at least a portion of the plurality of microfibers 204 includes at least one magnetic particle 224 at least partially embedded therein. The template further includes the plurality of cells 220 associated with the plurality of microfibers 204. In certain aspects, the plurality of microfibers 204 includes at least one first magnetic particle 224 at least partially embedded therein. In certain aspects, the plurality of cells 220 includes at least one second magnetic particle 244 at least partially embedded therein.

In certain aspects, the microfibers 204 include or are formed from PCL and at least a portion of the microfibers 204 include at least one FMP particle at least partially embedded therein.

According to various aspects of the present disclosure, a novel approach to templating capillary-scale microvasculature for supporting 3D tissues via magnetically assisted cell seeding is provided. Two commonly used techniques for producing polymeric microfibers to generate magnetic lattices including SEW and FEP are used. To efficiently endothelialize these highly porous lattices, ECs are endocytically loaded with SPIONs and subsequently magnetically attracted to FMPs embedded within lattice microfibers. Importantly, SPION magnetization of ECs do not impair viability or adherens junction assembly. In certain aspects, incorporating engineered capillaries within dense iPSC-CM-laden tissue, the endothelialized microchannels improve tissue survival. As compared to previously established methods for engineering capillary beds including angiogenesis and vasculogenic assembly, this top-down approach may yield readily scalable capillary beds over shorter time scales. This method has broad utility in engineering large, dense, 3D tissues that contain metabolically active parenchymal cells.

### EXAMPLE

Various embodiments of the inventive technology can be further understood by the specific examples contained herein. Specific examples are provided for illustrative purposes of how to make and use the compositions, devices, and methods according to the present teachings.

Reagents: All reagents are purchased from Sigma-Aldrich and used as received, unless otherwise stated

Lattice support device fabrication: Lattice support device molds are custom designed in Solidworks and 3D printed using a Formlabs Form 3 SLA printer with Grey v4 resin. Printed molds are treated with trichloro(1H,1H,2H,2H-perfluorooctyl)silane (TPS) to enable de-molding upon replica casting with Sylgard 184 polydimethylsiloxane (PDMS, 20:1 base:crosslinker). PDMS casts are additionally functionalized with TPS and used as stamps to emboss PDMS onto plasma-treated coverslips. To promote adhesion of collagen to the embossed PDMS lattice supports, the devices are treated with 5 v/v % (3-aminopropyl)trimethoxysilane in 100% ethanol for 24 hours, then dried and treated with 0.5 v/v % glutaraldehyde in Milli-Q water for 30 minutes. After treatment, devices are rinsed and dried in a vacuum oven overnight to remove residual glutaraldehyde.

Lattice fabrication via fiber electropulling (FEP) technique: Dry stocks of ferromagnetic neodymium iron boron (NdFeB) microparticles (FMPs) (Magnequench) are prepolarized by exposure to a 1.0 T magnetic field pulse. Polycaprolactone (PCL; Mn 80,000) is solubilized in chloroform in concentrations ranging from 10 to 25 w/v % and prepolarized FMPs are suspended at 4 w/v %. Solutions are loaded into a 1 ml syringe and collected using a modified version of a previously published dry spinning method where microfibers are pulled from solution via a rotating collection mandrel. PCL solution is expelled at 0.2 mL hr⁻¹ via syringe pump under a 3 kV voltage gradient. A grounded, rotating collector plate is loaded with the lattice support devices and translated at linear speeds of 28 cm s⁻¹, unless otherwise noted, to draw out solid PCL microfibers from solution. A linear actuator is used to translate the spinneret at a rate of 1 mL hr⁻¹ laterally along the mandrel axis to generate aligned arrays of microfibers. To generate multilayered microfiber lattices, lattice support devices are rotated 90 degrees in between sequential rounds of deposition.

Lattice fabrication via solution electrowriting (SEW) technique: PCL (Mn 80,000) is solubilized in chloroform in the concentration range from 10 to 30 w/v %, while the solutions containing FMPs are prepared as for the FEP method. For microfiber visualization, CellTracker Red CMTPX Dye (ThermoFisher) is added to the solution at 25 μg ml⁻¹. A repurposed fused deposition modeling (FDM) 3D printer (Ender 3 V2, Creality, China) is used for solution electrowriting (SEW) (alternatively termed near field electrospinning). The FDM printer's extruder is replaced with a custom-built holder for a blunt 22 G needle, while the printer's bed is replaced with an insulated copper plate serving as a charged collector surface. The needle is grounded and connected to the 1 ml syringe to extrude the polymer solution at 0.9 mL h⁻¹ feed rate, while the collector surface is connected to the high voltage power supply (Gamma High Voltage Research, USA). Fiber spacing and placement into lattices in SEW are controlled using custom gcode files. PCL fibers with and without magnetic particles are produced using a voltage gradient ranging from 2.5 to 5.5 kV (depending on polymer solution concentrations) and collected at a translation speed of 2500 mm min⁻¹. For cell-seeding studies, lattices are treated with 10 ng mL⁻¹ human plasma fibronectin (ThermoFisher) for 24 hours at 4° C. to facilitate cell adhesion.

Lattice magnetization and magnetic characterization: PCL lattices doped with FMPs are magnetized using a 1 T magnetic field pulse generated in a magnetorheology module in a TA Instruments Discovery Hybrid Rheometer HR30. To rewrite and align magnetic domains and define specific magnetization orientations, lattice microfibers are exposed either parallel or perpendicular to the imposed magnetic field. The magnetic properties of the NdFeB doped fibers are characterized at room temperature in a Lake Shore 7400 vibrating sample magnetometer. Magnetic hysteresis loops are acquired using a scanning field within the range of ±15 kOe to specific orientations, and paramagnetic backgrounds are subtracted. The magnetic remanences are measured at zero magnetic fields from the hysteresis loops.

Cell culture: Human umbilical vein endothelial cells (HUVECs; Lonza) or normal human dermal microvascular endothelial cells (MVECs; Lonza) are cultured on tissue culture plastic (TCP) dishes with standard endothelial growth medium-2 (EGM2; Lonza). Induced pluripotent stem cells (iPSCs) containing a GFP fusion tagged titin reporter (Allen Institute) are differentiated into cardiomyocytes (iPSC-CMs) using a previously established protocol. Briefly, iPSCs are cultured in mTeSR1 media (StemCell Technologies) and differentiated in RPMI 1640 media supplemented with 2 v/v % B27 minus insulin (ThermoFisher) and 1 v/v % GlutaMAX (ThermoFisher, 100×). Differentiation is initiated with the addition of 12 μM CHIR99021 at day 0 and followed by 5 μM IWP4 at day 3. iPSC-CMs are purified in RPMI lacking glucose and glutamine (Biological Industries) supplemented with 4 mM D/L-lactate and subsequently maintained in RPMI 1640 supplemented with 2 v/v % B27 (ThermoFisher) and 1 v/v % GlutaMAX. To generate a GFP-expressing ECs, cells are infected with lentivirus transducing pLJM1-EGFP (Addgene plasmid #19319). Lentivirus is produced in HEK293Ts using polyethylenimines-based transfection of 3rd generation viral packaging and transgene plasmids.

Cell magnetization: Polyvinylpyrrolidone (PVP) coated 8 nm super paramagnetic iron oxide nanoparticles (SPION; US Research Nanoparticles, Inc.) or NdFeB particles (Magnequench) are suspended in EGM2 at varying concentrations (50, 100, 200, 400, or 1000 μg mL⁻¹) and filtered through 10 μm sieves (pluriSelect) to remove large particle aggregates. Suspensions of magnetic particles in media are added to passage 3-8 ECs at 90% confluency and allowed to associate with cells for 16 hours prior to harvest.

Lattice seeding and culture: Magnetic particle-loaded ECs are washed twice with 1× phosphate-buffered saline (PBS) to remove non-endocytosed and non-adherent magnetic particles and detached from TCP with 0.05 v/v % Trypsin/EDTA solution. Cells are suspended in EGM2 and seeded onto fiber lattices at 200,000; 200,000,000; or 5,000,000 cells mL⁻¹. Cells are allowed to adhere to the PCL lattice for 24 hours prior to backfilling lattices with 4 mg mL⁻¹ rat tail type I collagen (Corning), prepared as previously described, or 10 mg mL⁻¹ fibrin. Briefly, collagen gels are prepared on ice with a reconstitution buffer (10 mM HEPES, 0.035 w/v % sodium bicarbonate, M199) and titrated to a pH of 7.6 with 1 M NaOH and Milli-Q water. For hydrogels encapsulating EC-seeded fibers without addition of parenchymal cells, gels are hydrated in EGM2 and media is replaced daily. Lipase is added at 1 U mL⁻¹ for 24 hours the day following encapsulation. Fibrin gels are prepared by mixing fibrin precursor solutions containing 10 mg mL⁻¹ fibrinogen from bovine plasma and 1 U mL⁻¹ bovine thrombin. For co-culture studies, iPSC-CMs are encapsulated in fibrin gels at 40,000,000 cells mL⁻¹ following pre-seeding with 5,000,000 cells mL⁻¹ ECs. Hydrogels containing iPSC-CMs are hydrated in RPMI 1640 media containing 2 v/v % B27 and 1 v/v % GlutaMAX supplemented with 5 μM Y-27632, 2 v/v % FBS, 25 ng mL⁻¹ phorbol 12-myristate 13-acetate (PMA), 50 ng mL⁻¹ vascular endothelial growth factor (VEGF; Peprotech), and 0.05 mg mL⁻¹ aprotinin. Hydrogel-encapsulated, cell-seeded lattices are maintained for an additional 24 hours prior to PCL microfiber degradation. To enzymatically degrade PCL lattices encapsulated in CM-containing hydrogels, 0.5 U mL⁻¹ lipase from Pseudomonas sp. is added to culture medium for 48 hours. Lipase is added to RPMI 1640 media containing 2 v/v % B27 and 1 v/v % GlutaMAX supplemented with 2 v/v % FBS, 25 ng mL⁻¹ PMA, 50 ng mL⁻¹ VEGF, 0.05 mg mL⁻¹ aprotinin, 250 mM HEPES, and 0.37 w/v % sodium bicarbonate. Lipase media is replaced after 24 hours. PCL is degraded after 48 hours of lipase exposure and media is replaced daily with RPMI 1640 media containing 2 v/v % B27 and 1 v/v % GlutaMAX supplemented with 2 v/v % FBS, 25 ng mL⁻¹ PMA, 50 ng mL⁻¹ VEGF, 0.05 mg mL⁻¹ aprotinin for the following 5 days.

Cell magnetization, viability, and proliferation assays: To assess magnetic particle loading into ECs, cells are passaged at a 1:8 surface area ratio into a 6-well TCP dish placed on top of a custom 3D printed plate holder housing 4 neodymium magnets (FIG. 6A). Magnetization efficiency was quantified by the number of cells adhering above the magnets compared to the number of cells adhering halfway between each magnet (5 mm). To determine cell viability in 2D, culture media is supplemented with Hoechst (1:1000; ThermoFisher) and propidium iodide (1:1000; ThermoFisher) for 20 minutes. To determine cell viability in 3D, hydrogels are stained after fixing. All cultures are then washed twice in PBS prior to fixation. Cell viability is determined from the percentage of propidium iodide or Phospho-Histone H2A.X positive nuclei of all DAPI- or Hoechst-stained nuclei. For proliferation assays, magnetized cells are seeded onto 18 mm coverslips or passaged twice prior to seeding onto coverslips. To quantify proliferating cells, EdU is supplemented in culture medium for the last 24 hours of culture. After fixation, EdU is fluorescently labeled following the manufacturer's protocol (ClickIT EdU, Life Technologies).

Bead perfusion: To assess lumenization of the vessels created after PCL microfiber degradation, the vessels are perfused with 4.18 μm fluorescent spheres (1:1000, Bangs Laboratory) diluted in PBS. Bead solution is added to one of the media wells after fixing the sample, the other media well is left empty to drive fluid flow through the patent channels.

Scanning electron microscope (SEM) imaging: HUVECs cultured without magnetic particles and with SPION or NdFeB particles are fixed with 4% paraformaldehyde (PFA). Samples are then dehydrated in graded concentrations of ethanol (30%, 60%, 90%, 100%) for 1 hour each. Terminal dehydration is performed in hexamethyldisilane which is evaporated in a vacuum chamber. After gold sputter coating using SPI-Module Carbon/Sputter Coater, SEM imaging is performed with a Thermo Fisher Nova 200 Nanolab SEM.

Fluorescence, Staining and Microscopy: Samples are fixed with 4% PFA for 1 hour at room temperature. To visualize the actin cytoskeleton and nuclei, samples are stained with FITC phalloidin (ThermoFisher) and DAPI (1:1000, ThermoFisher) for 1 hour for 2D samples or 8 hours for 3D samples at room temperature. For immunostaining, samples are additionally permeabilized in PBS containing Triton X-100 (5 v/v %), sucrose (10 w/v %), and magnesium chloride (0.6 w/v %) and blocked in 4% bovine serum albumin. Samples are then incubated in mouse anti-VE-cadherin (1:1000, Santa Cruz), mouse anti-podocalyxin (1:500, R&D Systems), Phospho-Histone H2A.X (1:200; #9718 Cell Signaling), or DAPI (1:1000) for 8 hours followed by Alexa-conjugated anti-mouse or anti-rabbit secondary antibodies for 8 hours each at room temperature. For UEA-1 staining, hydrogels are not permeabilized and blocked before staining with UEA-1 DyLight 649 (1:200; Vector). Fluorescent imaging is performed with a Zeiss LSM 800 laser scanning confocal microscope. Z-stacks are acquired with a 10× objective, unless stated otherwise. All images are presented as maximum intensity projections unless otherwise stated.

Statistical analysis: Statistical significance is determined by one-way or two-way analysis of variance (ANOVA) with Tukey test for multiple comparisons or two-sided Student's t-test where appropriate. Significance indicated by *p≤0.05, with alpha=0.05. Sample size is indicated within corresponding figure legends and all data are presented as mean±standard deviation. For two-group comparisons, a two-tailed Student's t-test is performed. GraphPad Prism v. 9.3.1 is used for data analysis and plotting. Sample size (n) and P-value are specified in the text of the paper or in the drawing descriptions.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method of producing a cellular support material, the method comprising:
introducing cells to a microfiber lattice, wherein the microfiber lattice has a three-dimensional structure comprising a plurality of fibers and a plurality of voids distributed between the plurality of fibers, wherein at least a portion of the plurality of fibers includes at least one first magnetic particle at least partially embedded therein and at least a portion of the cells includes at least one second magnetic particle at least partially embedded therein;
associating the cells with the plurality of fibers within the microfiber lattice;
encapsulating the microfiber lattice and associated cells with a hydrogel; and
removing at least a portion of the plurality of fibers.

2. The method of claim 1, further comprising fabricating the microfiber lattice having a three-dimensional structure comprising the plurality of fibers and the plurality of voids distributed between the plurality of fibers, wherein fabricating the microfiber lattice comprises solution electrowriting (SEW) or fiber electropulling (FEP) a fiber precursor material.

3. The method of claim 2, wherein the fiber precursor material comprises polycaprolactone (PCL).

4. The method of claim 1, wherein the plurality of fibers has an average diameter that is greater than or equal to about 5 micrometers to less than or equal to about 100 micrometers.

5. The method of claim 1, the method further comprising, prior to the associating, polarizing the microfiber lattice.

6. The method of claim 5, wherein the plurality of fibers is substantially aligned along a first direction and the polarizing the microfiber lattice comprises applying a magnetic field to the microfiber lattice in a second direction substantially orthogonal to the first direction.

7. The method of claim 5, wherein the plurality of fibers is substantially aligned along a first direction and the polarizing the microfiber lattice comprises applying a magnetic field to the microfiber lattice in a second direction substantially parallel to the first directions.

8. The method of claim 1, wherein the removing includes enzymatically degrading at least a portion of the plurality of fibers.

9. The method of claim 1, wherein the cells comprise endothelial cells (ECs).

10. The method of claim 1, wherein the first magnetic particle and the second magnetic particle are independently selected from the group consisting of: a ferromagnetic microparticle (FMP), a superparamagnetic iron oxide nanoparticle (SPION), or combinations thereof.

11. The method of claim 1, wherein the plurality of fibers comprise polycaprolactone (PCL), the first magnetic particle comprises a ferromagnetic microparticle (FMP), the cells comprise endothelial cells (ECs), and the second magnetic particle comprises a superparamagnetic iron oxide nanoparticle (SPION).

12. A cellular support material comprising:
a plurality of microchannels defined in a hydrogel matrix, wherein a first portion of the plurality of microchannels are aligned in a first direction; and
a plurality of cells embedded within surfaces defined by the plurality of microchannels, wherein at least a portion of the plurality of cells includes at least one magnetic particle at least partially embedded therein.

13. The cellular support material of claim 12, wherein the plurality of microchannels has an average diameter that is greater than or equal to about 5 micrometers to less than or equal to about 100 micrometers.

14. The cellular support material of claim 12, wherein the hydrogel matrix comprises a polysaccharide, a polypeptide, a synthetic polymer, or combinations thereof.

15. The cellular support material of claim 14, wherein the hydrogel matrix comprises a polypeptide selected from the group consisting of: collagen, fibronectin, gelatin, derivatives and combinations thereof.

16. The cellular support material of claim 12, wherein the plurality of cells comprise endothelial cells (ECs).

17. The cellular support material of claim 12, wherein the at least one magnetic particle comprises a superparamagnetic iron oxide nanoparticle (SPION).

18. A method of forming a microvasculature, the method comprising growing cells on the cellular support material of claim 12 until the microvasculature is formed.

19. A template for forming a cellular support material, the template comprising:
a plurality of microfibers defining a three-dimensional lattice and including a plurality of voids between the respective microfibers of the plurality, at least a portion of the plurality of microfibers includes at least one magnetic particle at least partially embedded therein; and
a plurality of cells associated with the plurality of microfibers, wherein at least a portion of the plurality of cells includes at least one magnetic particle at least partially embedded therein.

20. The template of claim 19, wherein the microfibers comprise polycaprolactone (PCL) and at least a portion of the plurality of microfibers comprise at least one ferromagnetic microparticle (FMP) at least partially embedded therein.
